(19) United States
(12) Patent Application Publication
(10) Pub. No.: US 2011/0091567 A1
(43) Pub. Date: Apr. 21, 2011

(54) TISSUE ORGANIZING STRUCTURE AND THERAPEUTIC METHODS

(76) Inventors: Sarah C. Crawford, Milford, CT (US); Herbert G. Potter, Hamden, CT (US); Tasino D. Herbert, Hamden, CT (US)

(21) Appl. No.: 12/672,383

(22) PCT Filed: Aug. 6, 2008
(86) PCT No.: PCT/US08/09419

§ 371 Date: Feb. 5, 2010

Related U.S. Application Data
(60) Provisional application No. 60/963,606, filed on Aug. 6, 2007.
(60) Provisional application No. 61/005,930, filed on Dec. 10, 2007.

(51) Int. Cl. A61K 35/12; C12N 5/09; A61K 36/00; A61P 35/00; A61P 35/02; C12N 15/87

(52) U.S. Cl. 424/520; 435/375; 424/725; 435/455

(57) ABSTRACT

A novel cellular component, tissue organizing structure, and methods for use in detection, diagnosis and treatment of disease is described herein. Particularly, clinical applications include tissue generation from individual cellular components, gene transfer therapeutics and its use as a novel target for cancer prevention and therapeutics. More particularly, methods for disease and cancer prevention, detection, diagnosis and treatment are provided.

### CROSS-REFERENCE TO RELATED APPLICATIONS

This application claims priority from U.S. Provisional Patent Application No. 60/963,606 filed Aug. 6, 2007, and U.S. Provisional Patent Application No. 61/005,930 filed Dec. 10, 2007.

### BACKGROUND

Despite many advances and current progress in the early detection of cancer, many malignant tumors are only first diagnosed at late stages of disease, long after the early events responsible for tumor development have given way to advanced, and often, widespread metastatic disease. This unfortunate circumstance both complicates cancer therapeutics and obfuscates a major objective of cancer biology: to explore the fundamental early stage events responsible for malignant tumor development and spread in the body. Research studies designed to recapitulate early stages in the natural history of solid tumor formation and development might be expected to afford a unique opportunity to assess fundamental parameters that underscore the abnormal growth behavior of genetically transformed cells and also to provide key insights into fundamental, but as yet, poorly understood oncogenic behaviors, including mechanisms of solid tumor formation, invasiveness and spread that distinguish cancer cells from their normal counterparts.

Identification of a novel cell-derived structure, hereinafter referred to as “tissue organizing structure”, or “TOS”, and methods for using TOS are described herein. TOS possesses unique functional capabilities whose clinical applications include, but are not limited to, tissue generation from individual cellular components, tissue morphogenesis, gene transfer therapeutics, and use as a novel target or therapeutic agent for cancer prevention and therapeutics.

The existence of these novel cellular components were previously unidentified in multi-cellular systems. These unusual structures were discovered during approximately thirty hours of live cell microscopy studies of tumor cells cultured under conditions designed to promote the formation of multi-cellular microscopic tumors. TOS play a direct role in orchestrating the process of solid tumor development in tumor cell lines from human malignancies of the colon, breast, lung and brain, which comprise some of the most common human cancers. The migrations and extensions of tumor masses resulting from TOS interactions suggest that they play a critical role in processes associated with tumor formation, with tumor spread and metastasis, and with fundamental components of cancer growth.

In addition, the first documentation is presented herein of properties associated with TOS comprising tissue organization, genetic recombination, cell transformation, phenotypic reversion and solid tumor behavior and structural organization that define the utility of TOS as bio-medically useful agents. The TOS have now been identified, their cellular origins and properties documented, and several clinical applications assessed which have not been done in any prior study. Additional biological parameters involve tissue morphogenetic and therapeutic properties associated with TOS activity enabling phenotypic cell transformation and the phenotypic reversion and induction of cell death in human cancers. Additional biological parameters describe TOS in primitive plant species that possess anti-cancer activity.

### DESCRIPTION OF RELATED ART

Over the past few years, increasing attention has been given to the heterogeneous components of the microenvironment in which cancers develop. Research studies have identified different classes of tumor cells including stem cell components that may serve as the drivers of malignant growth (see, for example, Wicha et al. Cancer Res. 66(4): 1883-90 (2006); Ferretti et al. Hum Reprod Update. 13(2): 121-41 (2007)). In addition, the tissue stroma and its effects on tumorgenesis have been an important object of study (see, for example, Tang et al. FASEB J. 21(14): 3777-85 (2007)). The role of abnormal vascularization associated with tumor-initiated angiogenesis has been shown to be instrumental in tumor spread and disease progression (see, for example, Timmins et al. Angiogenesis 7(2):97-103 (2004)). This complexity of the tumor microenvironment further complicates attempts to assess fundamental processes driving the process of solid tumor development and spread.

Historically, the tissue culture system has played a critical role in the study of cancer, in exploring cancer cell genetics, the transformed phenotype, chemical and viral carcinogenic mechanisms and preclinical therapeutic assessments. The studies conducted herein to explore the early stages of solid tumor development have utilized the tissue culture system to replicate various aspects of tumor cell behaviors that may be applicable to the natural setting in which tumors develop. Previous studies have documented that tumor cells derived from many diverse types of human malignancies can be induced to form solid tumor masses when deprived of a suitable substrate for attachment, thereby preventing monolayer culture growth in vitro. (see, for example, Sutherland, Science. 240: 177-184 (1988); Kunz-Schughart et al. Int. J. Exp. Pathol. 79: 1-23 (1998); Santini and Rainaldi, Pathobiology. 67(3): 148-57 (1999); and Santini et al. Crit Rev Oncol Hematol. 36(2-3):75-87 (2000)). Under these conditions, individual tumor cells spontaneously aggregate to form solid tumor masses that have been termed “multi-cellular tumor spheroids” (MTS). More recently, in vitro studies of MTS have been expanded to include additional components of the tumor microenvironment, including endothelial, immunological and connective tissue components in an attempt to define the relative contributions of each of these micro-environmental factors to solid tumor behavior. (see, for example, Drasdo and H6hme, Phys Biol. 2(3): 133-47 (2005) and Delsanto at al., Phys Rev Leff. 94(14): 148105 (2005)). Extensive studies of the in vitro solid tumor model suggest that it bears some similarity to microscopic solid tumor lesions in vivo and that cultured MTS respond to cancer therapeutics more similarly to solid tumors in vivo than do two-dimensional monolayer cultures of tumor cells. (see, for example, Song et al., In Vitro Cell Dev. Biol Anim. 40 (8,9):262-267 (2004)). More recently, tumor spheroids have been used to model chemotherapy responses to positron emission tomography (PET) imaging studies (see, for example, Monazzam A, et al. Application of the multi-cellular tumor spheroid model to screen PET tracers for analysis of early response of chemotherapy in breast cancer. Breast Cancer Research (9:45)(2007)).

There are many important theoretical models that have been proposed to explain the biological behavior of tumor cells relevant to the processes driving solid tumor formation, increases in mass, and invasive and expansive properties that may contribute to the metastatic phenotype. Many of these models are based on studies of solid tumor multi-cellular spheroids (MTS) in vitro. An early model proposed by Landry et al., used mathematical expressions to correlate tumor growth rate with specific measurable growth parameters, and predicted a linear expansion of spheroid diameter with time. (see, for example, Landry et al., Cell Tissue Kinet. 15 (6):585-94 (1982)).

A more recent model designed to explain solid tumor growth behavior involved the analysis of nutrient and biomechanical forces to assess the spatio-temporal growth dynamics of solid tumor cultures. (see, for example, Tindall and Please, Bull Math Biol 69(4): 1147-1165 (2007)). These computer-aided modeling studies suggested that a bio-mechanically mediated form of growth inhibition may be responsible for transitions from exponential to sub-exponential growth rates in large size tumor spheroids. Another recently proposed mathematical model was based on energy metabolism and reaction/diffusion equations to predict growth rate patterns and regions of quiescence within solid tumors that might have predictive value in establishing critical parameters of tumor survival. (see, for example, Venkatasubramanian et al., J Theor Biol. 242(2):440-53 (2006)). Additional studies of tumor cell growth dynamics associated with spheroid formation of a glioblastoma multiforme cell line based on physical measurements of spatio-temporal cell distributions, immunohistochemistry, and flow cytometry indicated that simple exponential growth kinetics could not account for the growth behavior characteristic of tumor spheroid formation (see, for example, Stein et al., Biophys J. 92(1):356-65 (2007)).

The growth pattern of microscopic glioblastoma multiforme was studied in vitro and observed that invasive tumor cells organize in branches. A model was proposed suggesting that homotypic attractions defined as the tendency of cells to travel previous paths coupled with heterotypic chemotaxis as the basis for the formation of these invasive zones (see, for example, Sander and Deisboeck, Phys Rev E Stat Nonlin Soft Matter Phys. 66 (5 Pt 1):051901 (November 2002) and Khain and Sander, Phys Rev Lett 96(18): 188103 (2006)). In addition, it has been proposed that volumetric growth dynamics in solid tumors may depend on surface extension parameters based on the “‘universal scaling law” (see, for example, Doisboeck et al., Med Hypotheses. 67(6); 1338-41 (2006)). They propose that cancer invasion should occur via a few branches of mobile cells and that this surface expansion may permit the diffusion of nutrients to permit tumor establishment in the absence of neoangiogenesis. Scaling techniques utilized to assess the fractal nature of developing tumors from cell lines in vitro and also in vivo have shown that both groups of tumors display similar growth dynamics which correspond to molecular beam epitaxy (MBE) universality class. (see, for example, Bru et al., Biophys J. 85(5):2948-61 (2003)). MBE dynamics are characterized by a linear growth rate, limitation of cell proliferation to the edge or border of the tumor and the surface diffusion of cells at the growing edge, in place of exponential growth behavior. The authors proposed that a significant determinant of cell movements or diffusion at the tumor border involves the intrinsic requirement of the tumor for space in competition with normal host tissue, perhaps more important than nutrient or other factors in driving this expansion. Their experimental data suggest that cell surface diffusion represents a critical determinant governing the pattern of tumor growth. These arguments provide conceptual support for TOS being the intrinsic cellular source of tumor expansion and invasiveness that permits the development of this system consistent with this theoretical framework.

Recent studies of solid tumor micro-environmental associations in breast cancers and other solid tumors have led to the proposed concept of an epithelial-mesenchymal transition (EMT) as the basis for many of the morphological cell structure changes associated with oncogenesis of epithelial cells to abnormal cells of fibroblastic morphology. (see, for example, Kokkinos et al., Cells Tissues Organs. 185 (1-3): 91-203 (2007)). Moreover, genetic analyses of normal stromal tissue in contact with malignant colon cells suggest that EMS may also affect the tumor microenvironment and promote disease spread as the result of genetic induced in-tumor associated stroma. (see, for example, Sheehan et al., Oncogene. 27(3):323-31 (Jan. 10, 2008)).

Our findings suggest a mechanism by which this proposed “crosstalk” might occur, in that TOS fusions with normal stromal cells surrounding a developing tumor could induce genetic changes consistent with these observations. Research data supportive of this model conducted in this laboratory comprises: (1) phenotypic transformation of normal cultured lung cells (cell line WI38) to a highly aggressive malignant cell type by cell-free transfer of TOS isolated from a malignant lung tumor cell line (cell line H1299) (2) phenotypic reversion and cell death induction of malignant lung tumor cell line (cell line H1299) by normal lung fibroblasts (cell line WI38); and (3) morphological evidence of EMT-like tissue transformations induced by TOS from breast tissue incubated with glial brain tissue.

Additional theories have suggested that cell fusions between genetically transformed “pre-malignant” cells and tissue stem cells may mediate horizontal gene transfers responsible for early stage oncogenesis (see, for example, Tysnes and Bjerkvig, Biochem. Biophys. Acta. 1775(2): 283-97 (June, 2007)). However, the observations herein provide direct evidence that cell fusions and horizontal genetic recombinations occur frequently in early stages of solid tumor formation in vitro and that TOS cell components are the structural vehicle responsible for these events.

Other journal publications include a recently published study by Nedawi et al (2008) that discusses the role of secreted membrane vesicles called exosomes derived from mast cells in inducing plasminogen activator inhibitor type I activity in endothelial cells. (see, for example, “Intercellular transfer of the oncogenic receptor EGFRvIII by microvescicles derived from tumor cells”, Al-Nedawi et al, NCB 10(5) (May 2008)). The findings in the Medawi study differ substantively from the TOS discovery in that the researchers conducted the study using conventional exosomes, performed the study as an in vitro experiment with speculative biological significance in an intact cellular system, and postulated that the resultant phenotypic changes were based on passive protein transfer rather than resulting from a stable morphogenetic transformation, an activity that is documented in TOS particles. An earlier study by Bergsmedh et al (2001) documented the horizontal transfer of oncogenes by apoptotic bodies. (see for example, “Horizontal Transfer of Oncogenes by Uptake of Apoptotic bodies” Bergdmedh et al., PNAS 98(11) (May 2001)). This study also differed substantively from the TOS finding in that apoptotic bodies obtained from dead cells were used as a source of gene transfer. In addition, genetically altered cell lines were used as a source of transfected genes for horizontal transfer. Most importantly, the gene transfer and associated phenotypic effects were unstable, inducing a transient phenotypic alteration that resulted in loss of the transferred genes within four weeks in culture. In contrast, the TOS activities are associated with a stable morphogenetic transformation resulting in the production of stable transformed cell lines that have been maintained for many months in culture. Yet another study, del Conde et al Blood 106 (March, 2005) documented the capacity of monocyte/macrophage derived microvesicles containing tissue factors to fuse with activated platelets to initiate platelet coagulation. The demonstrated biomedical activities of TOS illustrate the clinical potential of, and need for, TOS as a diagnostic and treatment agent in human health and disease.

The related art does not address the identification of TOS nor its biomedical significance as identified herein. The identification of TOS and the methods of utilization are a significant departure from the prior art in that novel processes have been identified by which cancer cells organize in culture to form three-dimensional solid tumors that involves cell-derived membrane-bound structures (TOS) that orchestrate the process of solid tumor formation and expansion.

An embodiment of the process for TOS mediated solid tumor development comprises a functional resemblance to other biological systems, which thus has relevance to cancer biology, including the blastocyst stage of embryogenesis which appears to resemble the fission-like process associated with TOS formation; schizogamy, which is a rapid cell division of the malarial parasite in the liver, and resembles the fission-like reproduction of TOS; and simple genetic exchange mechanisms characteristic of unicellular organisms. These comparisons may be significant since oncogenesis may recapitulate in early stage embryogenic stem cell developmental processes, and may also occur in a parasitic mode reminiscent of primitive eukaryotic parasitic systems.

### SUMMARY

A unique cellular component with unprecedented and previously undescribed properties with direct clinical and biomedical therapeutic applications is described herein. No previous study has identified or characterized the activities or biomedical uses of the unique cellular component designated herein as “tissue organizing structure” or “TOS”. Nor has any previous study documented the specific activities of the TOS applicable to the prevention and treatment of diseases, including cancer, as well as tissue engineering and genetic engineering for the treatment or prevention of diseases.

### Identification and Characterization of TOS

The spatio-dynamic data that were obtained from microscopic observations of live tumor imaging and staining for structural assessment indicated that one embodiment of TOS formation is as a “bud” from the surface membrane of tumor cells. Individual TOS budding is generally induced by cell detachment from substrate or from other cell contacts, indicating that an important stimulus for TOS production is the abrogation of cell-to-substrate or cell-to-cell contacts and associated signaling pathways. Thus TOS appear to be membrane-delimited vesicles that frequently contain genetic material based on their staining properties, although empty vesicles that do not contain genetic material have also been observed.

In another embodiment, a plurality of TOS are produced by a novel autonomously driven reproduction mechanism occurring via rapid cell fission. TOS-cell interactions were observed to induce the formation of large numbers of TOS vesicles within the targeted cell, which was subsequently associated with cell lysis and release of the TOS vesicles. This TOS-induced production of TOS occurs via a fission-like process in which a cell is induced by TOS-mediated activity to undergo a rapid morphogenesis to what is observed to be a plurality of TOS within the cell, or “bag of TOS”, that are subsequently released by the rupture or lysis of the cell membrane.

One or more methods of extracting TOS from cells and enabling isolation and purification of TOS as a cell-free extract are provided herein. A Method utilizing cells of human origin comprises generally culturing cells to stimulate TOS production, treating the cells with trypsin to accumulate the TOS, the media and cells are then removed from the culture dish to isolate the TOS, the TOS are scraped off the bottom off the dish and re-suspended in culture medium for removal in pure form in the absence of cellular material. A method utilizing plant cells comprises generally preparing plant material (leaves and stems) and then culturing as above to isolate and purify TOS.

One or more methods of identification of TOS are provided herein generally comprising culturing cancer cell lines, utilizing videography and/or photomicroscopy to record tumor chronology and development, and identification of TOS by direct observation and/or selective staining techniques.

One or more methods of detection of TOS in a biological sample are provided herein. In one embodiment, TOS may be routinely identified in cells in culture following trypsinization and replating. This method of cell culture manipulation for routine passage and maintenance induces the spontaneous budding of TOS vesicles from the surface of the cultured cell. Confluent substrate attached cells are not generally associated with TOS formation or release from cultured cells.

The mechanism of action of these unique TOS cellular entities associated with therapeutic applications has not been previously described or identified. TOS were observed to exist in one of two forms: either as “empty” membrane-bound vesicles or as vesicles containing genetic material. The genetic material was observed to be acquired by the empty vesicles following their entry into tumor cells, as this was often followed by their subsequent emergence with newly acquired contents comprised of genetic material. The TOS were also observed to fuse with one another and appeared to exchange genetic material before dissociating and moving apart. Overall, TOS were observed to be extraordinarily plastic with respect to their ability to fuse with one another to form doublets or even triplet complexes, and to enter tumor cells. This plasticity extends to their genetic contents as well, in that TOS were observed to exchange genetic information with other TOS, and also with cells.

TOS appear to effect their environment by one or more methods of rapid mobility of the TOS, and by methods of exchange or transfer of genetic material. Once produced, the TOS demonstrate (1) the capacity to fuse with other TOS and recombine genetic material before separating, an event that was sometimes associated with cell divisions in duplicate or triplicate. TOS then demonstrate the ability to act on tumor cells by (2) entering tumor cells by membrane fusion, and then inducing rapid morphological, changes in the host tumor cell, including but not limited to morphogenesis to the “TOS bags” described above.

The ability of TOS to effect significant tumor changes was demonstrated by their rapid mobility, where TOS were observed to spread rapidly throughout the culture by several means, including the use of extra-cellular matrix (ECM) fibers produced by the tumor cells as pathways from one cell mass to another, and by organizing into tubular masses following their release from tumor cells to form “TAS tubules” as connectors linking various areas of the tumor mass. Thus TOS-tumor cell interactions were associated with tubular extensions of solid tumor masses resulting from the TOS interactions

Pharmaceutical compositions utilizing TOS to affect biological processes may be provided. An embodiment of a pharmaceutical composition for the treatment of a mammalian disease comprises purified TOS having a therapeutic effective activity, combined in a mixture with a pharmaceutically acceptable carrier. A composition is said to be a “pharmaceutically acceptable carrier” if its administration can be tolerated by a recipient patient. For purposes of therapy, compositions comprising TOS and a pharmaceutically acceptable carrier are administered to a patient in a therapeutically effective amount. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995). The choice of a pharmaceutically acceptable carrier, including a physiologically acceptable agent, depends, for example, on the route of administration of the composition by any number of routes known in the art.

In a further embodiment, packaged pharmaceuticals comprising TOS therapeutic anti-cancer activity are provided herein. In one embodiment, the packaged pharmaceutical comprises: (i) an agent comprising a therapeutically effective amount of TOS that is toxic to human tumorigenic cells; and (ii) instructions and/or a label for administration of the agent for the treatment of patients having cancer. In another related embodiment, the packaged pharmaceutical comprises: (i) a therapeutically effective amount of an agent that that interacts with a cellular component that interacts with TOS; and (ii) instructions and/or a label for administration of the agent for the treatment of patients having cancer.

### Therapeutic Indications for TOS

The assessments of the biological functions of TOS indicate it as a therapeutic target in the treatment of disease in biological organisms, and also as a preventive agent to prevent or halt the development of disease, including but not limited to early stage cancer and metastatic disease. The assessments include but are not limited to discovery of TOS in plant species that display therapeutic anti-cancer activity as demonstrated in pre-clinical assessments, the experimental determination that TOS derived from normal tissue can induce phenotypic reversion and cell death in tumor cells of the same tissue type, and the experimental determination that TOS can induce morphogenetic transformation of target tissue to cells resembling the tissue origins of the transforming TOS particles. Moreover, the diverse biological functions indicate TOS as a tool in the development of novel methodologies for tissue engineering, gene transfer therapeutics and genetic engineering.

In a further embodiment, TOS derived from normal tissue may be utilized as a therapeutic agent in the treatment of disease. For example, TOS derived from normal tissue has been utilized to induce phenotypic reversion and cell death in tumor cells of the same tissue type. The fact that TOS derived from normal tissue can induce phenotypic reversion and cell death of malignantly transformed cells demonstrates therapeutic value of cell-derived TOS in the detection and treatment of cancer via a novel biological therapeutic approach.

In a further embodiment, a method is provided utilizing TOS to treating disease in an animal comprising the steps of providing a source of therapeutic TOS, prepared either by culturing the cells of the animal under conditions that promote TOS formation, or from another cellular source as appropriate based on the nature of the disease condition, followed by TOS purification, then administering the composition to the animal, or to cells derived from the animal, to induce the morphogenetic correction of diseased tissue to produce functional tissue.

### TOS in Genetic Information Transfer and Genetic Engineering

TOS has been demonstrated to enables somatic (horizontal) genetic exchange, cell morphogenesis and intercellular communication as demonstrated herein. TOS exist at least as either as membrane-bound vesicles without genetic material, or as vesicles containing genetic material. Empty vesicles acquire genetic material after entry into cells. TOS may also fuse with one another, exchange genetic material, dissociate and move apart. TOS may fuse to form at least doublet or triplet complexes.

In a further embodiment, a method is provided for detecting a genetic abnormality in a Patient, comprising, obtaining tissue from a patient, culturing the tissue in vitro to produce TOS from the cells of the patient, isolating the TOS, utilizing the TOS in cellular transformation studies, assessing the phenotypic and genetic results of the TOS cellular transformation studies, and identifying the specific abnormal genes responsible for disease.

In a further embodiment, one or more methods are provided utilizing TOS for the introduction of one or more genes into a cell. In one embodiment, TOS may be genetically engineered in vitro to incorporate specific genes for therapeutic transfer. In a further embodiment, TOS may be purified in vitro from cells containing functional genes of interest. Gene transfer to recipient cells may be achieved in culture using the ex vivo methods provided herein. Gene transfer in a recipient may be affected by TOS administered to a recipient via a pharmaceutically acceptable composition wherein TOS comprising one or more genes of interest is combined in a mixture with a pharmaceutically acceptable carrier to comprise a pharmaceutically useful composition for administration to a recipient according to known methods.

In a further embodiment, a method is provided utilizing TOS to transfer one or more genes between cells comprising the steps of (1) selecting one or more cells from a patient or other source having genetic material expressing phenotype characteristics of interest, (2) transferring the genetic material to TOS by incubating the selected cell(s) with a TOS composition, (3) introducing the TOS-genetic material product into a recipient cell, wherein the gene product exhibits the specific phenotype characteristics of the source.

TOS represent an important potential tool for gene therapy. TOS are useful vectors for gene delivery as they efficiently and rapidly interact with cell surfaces, and are readily internalized to elicit rapid morphogenetic effects on the target cell. TOS represent an important tool for gene delivery in that they induce genetic recombination between their genetic contents and the host cell chromosomes to achieve a stable genetic and phenotypic effect in the target cell. TOS may be produced and purified to any desired amount or concentration utilizing the methods provided herein. A method of gene therapy utilizing TOS comprises the steps of providing therapeutic genetic material within TOS by the methods disclosed herein, providing a mixture of the TOS and a pharmaceutically acceptable carrier, and then administering the mixture to a subject in a therapeutically effective amount to deliver the TOS-gene product and produce a phenotypic response.

More particularly, a method of gene therapy for muscular dystrophy utilizing TOS as a gene delivery tool would comprise the isolation of myoblasts from a patient with muscular dystrophy, eliciting TOS-mediated gene transfer of the dystrophin gene to the patient to affect the associated phenotypic conversion of the diseased myoblast to a dystrophin-producing cell. An embodiment of the method comprises transferring the dystrophin genetic material to TOS by incubating dystrophin phenotypic cells with a TOS composition, isolating the TOS-dystrophin product, providing a mixture of the TOS-dystrophin product in a therapeutically effective amount with a pharmaceutically accepatable carrier, administering the TOS-dystrophin mixture to a recipient, wherein the gene product exhibits the specific phenotype characteristics of the source.

In a further embodiment, a method is provided utilizing TOS as a tool for bioengineering of recombinant genotypes based on the facility with which these structures initiate recombination among themselves and with host cells in culture. That TOS enter cells by fusion and exit by fission, or budding, facilitates their use as gene delivery systems.

### Therapeutic Aspects of TOS in Disease and Cancer

There are many potential implications of the involvement of TOS in de novo solid tumor development in furthering our understanding of how cancer develops in its earliest stages to acquire an invasive, metastatic, and genetically unstable phenotype. TOS are novel structural cell-derived entities which have been demonstrated herein to be responsible for multiple aspects of solid tumor development. In another aspect, TOS enables genetic instability in tumors via novel forms of cell fusion and somatic recombination. In another aspect, tumor spread via formation of TOS tubules as conduits facilitates TOS migration, thereby enabling the further spread of TOS, increasing tumor network surface area, increasing cell-to-cell contact in the absence of angiogenesis, and thereby permitting tumor spread without vascularization. In another aspect, TOS enables cell-to-cell contact and tumor cell mass migration to generate solid tumors. In another aspect, TOS has been demonstrated to mediate tumor/stroma micro-environmental interactions associated with proposed epithelial-to-mesenchymal transitions (EMTs), and tumor/stem cell interactions. In another aspect TOS represents a preventive and therapeutic anti-cancer target based on the role in solid tumor formation and spread, analogous to the processes of invasion and metastasis. TOS has been utilized to induce the morphological and physiological transformation of normal tissue into tumors. Thereby demonstrating tumor-derived TOS as an important clinical therapeutic target in the treatment and prevention of cancer. In another aspect, TOS acts as a therapeutic target in that the vascular spread of TOS enables tumor metastases transforming normal tissue at diverse systemic sites remote from the origin of the primary tumor, with the result of metastatic disease. Thus, TOS from cancer cells represent an important therapeutic target in clinical medical treatment of systemic disease.

In a further embodiment; TOS may be utilized to detect cancer in a patient. This test could be done rapidly using previously described TOS purification protocol. The biopsy tissue-derived TOS could then be used in a rapid transformation assay in vitro to assess the ability of the TOS to elicit transformation of standard cultured cells. This test would supplement current histological assessments of tissue malignancy by providing a functional assay of malignant potential in a convenient in vitro system. An embodiment of a method comprises the steps of obtaining a tissue or other biological sample that will elicit the cancer phenotype from a patient, extraction and isolation of the TOS from the patient sample containing the cancer phenotype material of the source cancer cells, culturing the TOS-cancer phenotype in vitro with standard culture cells capable of being transformed by the TOS-cancer phenotype, and assessing the effect on the culture cells to determine if the phenotype of the source cells is present.

In a further embodiment, a method is provided to identify TOS mediated therapeutic targets in cancer, wherein solid tumors enabled via the TOS mechanism of solid tumor formation and spread, identifies these novel TOS structures as important therapeutic and preventive anti-cancer targets. Based on the documented capacity of TOS to induce the phenotypic reversion of malignant transformed cells in culture, the TOS mediated therapeutic targets may be identified directly by assessing the genetic composition of therapeutic TOS to identify the genes responsible for this therapeutic effect. An embodiment of a method to identify a TOS mediated therapeutic target comprises the steps of culturing purified TOS with recipient cancer cells, assessing the phenotypic response of the cultured cancer cells to determine therapeutic effect of TOS, isolating the DNA from the therapeutic TOS responsible for achieving the therapeutic effect, and analyzing the DNA sequence to identify the gene(s) responsible for mediating therapeutic effect.

TOS represent a potentially useful tool to prevent disease or modulate a disease target due to its capacity to induce morphogenetic tissue transformation. Methods are provided utilizing TOS to treat or prevent disease (e.g., cancer or HD) by modulating the function (e.g., activity or expression) of a target (cellular component) that is identified as provided herein. To illustrate, if a target is identified to promote tumor growth, a TOS therapeutic agent can be used to inhibit or reduce the function (activity or expression) of the target. Alternatively, if a target is identified to inhibit tumor growth, a TOS therapeutic agent can be used to enhance the function (activity or expression) of the target. A TOS therapeutic agent may include, but is not limited to, TOS isolated from non-diseased cells, diseased cells or abnormal cells. An embodiment of a method of preventative treatment of a disease comprises the steps of purifying TOS from normal functioning tissue, providing a mixture of the TOS and a pharmaceutically acceptable carrier, administering the mixture ex vivo or in vivo to a subject in a therapeutically effective amount, and correcting genetic effects that pose risk for disease development. An embodiment of a method of treatment of a disease comprises the steps of purifying TOS from normal functioning tissue, providing a mixture of the TOS and a pharmaceutically acceptable carrier, administering the mixture in vivo to a subject in a therapeutically effective amount, and inducing the death of abnormal cells.

In a further embodiment, methods are provided to treat or prevent cancer. TOS derived from normal tissue can be utilized as therapeutic agents in the treatment of cancer based on studies demonstrating that TOS from normal cells are capable of inducing phenotypic reversion of transformed tumor cells in vitro with the result of the induction of cell death. Cancer diseases include, but are not limited to, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma.

In one embodiment, a method of treating or preventing cancer in an individual comprises administering to the individual a therapeutically effective amount of a TOS composition that is selectively toxic to a tumorigenic cell. The methods comprising the steps as stated above for treatment or prevention of a disease. By “therapeutically effective amount” is meant the amount of the TOS composition administered that is sufficient to elicit the desired therapeutic effect (e.g., the death of a neoplastic cell). It is generally understood that the effective amount will vary according to the weight, sex, age, and medical history of the subject. Other factors which influence the effective amount may include, but are not limited to, the severity of the patient's condition, the disorder being treated, the stability of the TOS composition, and if desired, another type of therapeutic agent being administered with the TOS composition. A larger dose, or a plurality of doses, can be delivered by multiple administrations of the TOS composition. Methods to determine therapeutic efficacy and dosage are known to those skilled in the art For example, therapeutic efficacy may be assessed by standard methods of patient responses to radiation/chemotherapy. In addition, a pre-treatment assessment of potential TOS therapeutic effects may be demonstrated on biopsy tumor material cultured with TOS as stated herein.

Plant Derived TOS and Therapeutics

The discovery of TOS in a primitive plant species implicates this class of cellular structures as a diverse and widespread occurrence in eukaryotic multi-cellular systems with implications for activity in the basic processes of embryogenesis, tissue and organ system development, and tissue repair mechanisms. The novel biological activities of TOS derived from plant species have clinical applications. TOS derived from plant species display therapeutic anti-cancer activity as demonstrated herein by pre-clinical assessments. More particularly, TOS derived from primitive plant species (fern) demonstrated cytotoxicity. TOS derived from a fern species was found to display therapeutic properties involving the induction of cell death in a human malignant tumor cell line (glioblastoma multiforme). The cell-killing effect of the plant TOS was specific for the malignant tumor cells, a selective cytotoxic response, as comparable treatment of normal cells growing in culture (diploid lung fibroblast W138 cell line) did not produce cytotoxic effects.

In a further embodiment, the biological functions of plant TOS with therapeutic anti-cancer activity suggest that these structures play an important role in plant defense mechanisms that target diverse cellular systems and adds importantly to the spectrum of TOS activities with potential therapeutic applications. These unique cellular entities may represent a critical cellular component associated with basic processes of metazoan evolution, thereby contributing to a fuller understanding of basic biological processes critical to the structural and functional organization and development of the multi-cellular system. Moreover, the experimental manipulation of TOS may afford tremendous novel opportunities for developing new therapeutic strategies for the treatment of diseased human tissues.

In a further embodiment, methods are provided herein utilizing plant derived TOS to treat or prevent cancer. In one embodiment, a method of treating or preventing cancer in an individual comprises administering to the individual a therapeutically effective amount of a plant derived TOS composition that is selectively toxic to a tumorigenic cell. The methods comprising the steps as stated above for treatment or prevention of a disease.

### TOS and Tissue Engineering

TOS in normal cells enables tissue morphogenesis, development, and embryogenesis. TOS may be utilized as a tool for bioengineering of diverse tissue types based on the observed activities of TOS in the elaboration of complex tissue-like structures and in the induction of major morphogenetic changes in target cells.

In a further embodiment, TOS induces morphogenetic transformation of target tissue to cells resembling the tissue origins of the transforming TOS. The genetic and morphological transformations associated with TOS, i.e., producing tissue-specific morphogenesis, has profound implications for the treatment of a broad spectrum of diseases resulting from genetic dysfunction, tissue damage or injury. Moreover, the observed morphogenetic properties of tissue-specific TOS in inducing phenotypic responses in target cells, that are associated with conversion to the tissue type from which TOS originated, demonstrates therapeutic value in the morphogenetic transformation of diseased tissue to a normal phenotype.

TOS enables transfer of genetic material between cells, as discussed above. TOS activity has a profound effect on gene expression consistent with morphogenetic transformation, and the stable transfer of genetic material by means of genomic recombination with recipient cells. The controlled regulatory mechanism directing patterns of gene expression consistent with specific tissue types intrinsic to the mechanism of action of TOS particles demonstrates the corrective genetic remodeling of diseased human tissues of all types using TOS based therapeutics.

In a further embodiment, methods utilizing TOS are provided for tissue engineering or tissue repair, comprising the steps of (1) providing a source of tissue genetic material, (2) culturing the tissue genetic material with purified TOS from normal functioning cells, or other cells chosen for specific properties, to induce TOS to incorporate the cellular genetic material to be utilized as a TOS agent carrying a tissue specific phenotype of the source, (3) purifying and isolating the TOS agent, (4) culturing the TOS agent with target tissue to promote cellular differentiation of the target tissue to the tissue specific phenotype of the TOS agent source, (5) the differentiation being effective for tissue repair. In a further embodiment, a method for producing cells with a tissue-specific phenotype, useful for autogenous tissue repair, comprises the steps of: (1) obtaining tissue from an intended patient; (2) converting the tissue cells into repair and/or replacement cells by culturing with TOS as stated above, the converted cells having effective tissue-specific phenotype activity of the patient.

In a further embodiment, a tissue repair precursor composition, comprising a biocompatible matrix and a dense cell mass growing on the matrix exposed to a culture medium, the dense cell mass being converted to the tissue-specific phenotype exhibiting cell appropriate morphology, the culture medium including at least one TOS agent that promotes tissue-specific phenotype differentiation.

These and other aspects will become evident upon reference to the following detailed description:

### DESCRIPTION OF THE DRAWINGS

FIG. 1 is a diagram of an embodiment of TOS formation.

FIG. 2 is a photomicrograph (1000×) of purified TOS.

FIG. 3 are embodiments of TOS formation: FIG. 3a is a diagram of an embodiment of TOS formation. FIGS. 3b-3d are photomicrographs (1000×) of TOS formation.

FIG. 4 are embodiments of TOS cell-to-cell contact: FIG. 4a is a diagram of an embodiment illustrating TOS cell-to-cell contact. FIGS. 4b-4f are photomicrographs (1000×) of cell-to-cell contacts.

FIG. 5 is a photomicrograph (1000×) of WI38 normal diploid fibroblasts with no TOS control.

FIG. 6 are photomicrographs (1000×) of W138 normal diploid fibroblasts after exposure to TOS.

FIG. 7 are photomicrographs (1000×) of normal lung diploid fibroblasts prior to TOS transformation.

FIG. 8 are photomicrographs (1000×) of TOS prepared from mouse endothelial cells that contain the GFP (green fluorescent protein) gene.

FIG. 9 is a photomicrograph (1000×) of TOS particles stained with acridine orange.

FIG. 10 is a diagram of an embodiment of the TOS cell transformation process.

FIG. 11 is a photomicrograph (1000×) of TOS transformed lung fibroblasts used for control.

FIG. 12 is a photomicrograph (1000×) of transformed TOS lung cells exposed to normal TOS 24 hours.

FIG. 13 is a photomicrograph (1000×) of TOS transformed lung fibroblasts exposed to normal lung TOS after ten days.

FIG. 14 is a photomicrograph (1000×) of TOS transformed lung fibroblasts exposed to 233 normal TOS 24 hours.

FIG. 15 is a photomicrograph (1000×) of TOS transformed lung fibroblasts exposed to 2× normal TOS after 10 days.

FIG. 16 is a diagram of an embodiment of cellular reversion by TOS.

FIG. 17 is a photomicrograph (1000×) of normal TOS extracted from primitive plant (fern).

FIG. 18 are photomicrographs (1000×) of TOS transformed lung fibroblasts exposed to plant TOS at approximately two weeks.

FIG. 19 is a photomicrograph (1000×) of normal diploid fibroblasts exposed to plant TOS at approximately two weeks.

FIG. 20 are embodiments of TOS tubule formation: FIG. 20a is a diagram of an embodiment illustrating TOS tubules formation; FIGS. 20b-g are photomicrographs (1000×) of TOS tubule formation.

FIGS. 21a-i are photomicrographs (1000×) illustrating TOS bodies in solid tumor architecture.

FIGS. 22a-c are photomicrographs (1000×) illustrating TOS isolates in culture.

FIG. 23 is a sequential series of photomicrographs (1000×) depicting a TOS structure formation.

FIG. 24 is video frame showing TOS-mediated movement in NCI-H1299 lung carcinoma cells.

FIG. 25 is a video frame showing TOS budding from the surface of a glioblastoma GBM cell.

FIG. 26 is a video frame showing TOS-associated tubules and cell extensions in GBM cells.

FIG. 27 is a video frame showing TOS attaching to a NCI-H1299 lung carcinoma cell.

FIG. 28 is a video frame showing TOS-induced morphological transformation of GBM cell to “TOS bag”.

FIG. 29 is a video frame showing empty TOS interacting with dense TOS and cell membranes at the border of solid tumor cell.

FIG. 30 is a video frame showing TOS-TOS genetic exchange.

FIG. 31 is a video frame showing with cell migration and fused TOS with chromosome interactions.

FIG. 32 is a video frame showing shows early stage solid tumor formation and dense areas which often serve as initiation sites for TOS formation.

FIG. 33 is a video frame showing tubules and extensions associated with TOS activity in GBM solid tumor.

FIG. 34 is a video frame showing TOS tubules emerging from cell body serving as conduits for TOS migration.

FIG. 35 is a video frame showing individual TOS traveling through a tubule.

FIG. 36 is a video frame showing TOS tubule in association with a tumor cell membrane.

FIG. 37 is a video frame showing TOS in various stages of budding from a cell membrane.

FIG. 38 is a video frame showing TOS-mediated cell-to-cell interactions.

FIG. 39 is a video frame showing fused TOS in genetic exchange.

FIG. 40 is a video frame showing TOS budding and release.

### DETAILED DESCRIPTION

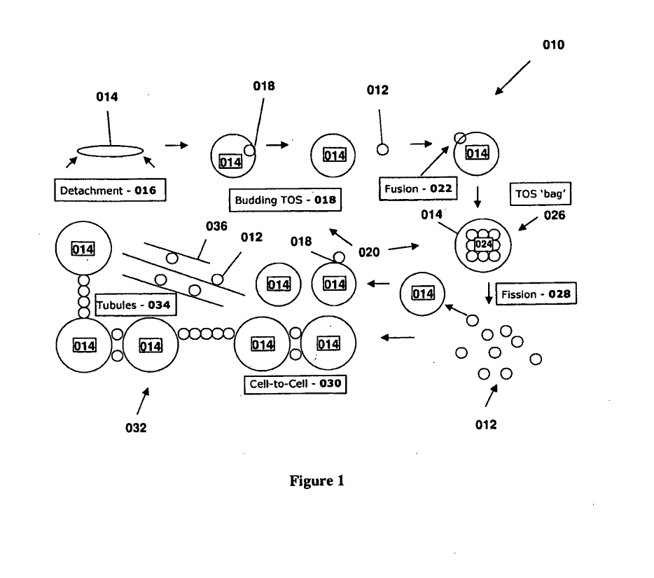

Studies of the earliest stages of solid tumor mass formation in vitro revealed that this process is critically linked to the formation of cell-derived membrane-bound structures, or Tissue Organizing Structures (TOS) as shown in FIG. 1. These previously unidentified structures affect aspects of tumor cell behavior associated with solid tumor development in vitro. The spatio-dynamic data that were obtained from microscopic observations of live tumor imaging and staining for structural assessment indicate that TOS can form as “buds” from the surface membrane of tumor cells, but more commonly appear to form via a fission-like process in which a tumor cell is induced to undergo a rapid morphogenesis to what appears to be a plurality of TOS within the cell, or a “bag of TOS” as discussed herein, that are subsequently released by the rupture or lysis of the tumor cell membrane (FIG. 1).

TOS appear to be membrane-delimited vesicles that frequently contain genetic material based on their staining properties, although empty vesicles that do not contain genetic material have also been observed. Other properties of TOS include rapid mobility and the capacity to fuse with other TOS and recombine genetic material before separating, an event that is sometimes associated with cell divisions in duplicate or triplicate. TOS enter tumor cells by a process that appears to occur by membrane fusion. The TOS then induce rapid morphological changes in the host tumor cell, including morphogenesis to “TOS bags”.

TOS were observed to spread rapidly throughout the culture by several means, including the use of extracellular matrix (ECM) fibers produced by the tumor cells as pathways from one cell mass to another. In addition, TOS were capable of organizing into tubular masses following their release from tumor cells to form “TOS tubules” as connectors linking various areas of the tumor mass. These observations indicate that these unique cell-associated structures are highly pleomorphic with respect to their structural parameters and extraordinarily diverse functionally with respect to their ability to interact with each other, with normal or tumor tissue, and to exchange genetic information.

Reference is now directed the Figures which utilize videography, photomicroscopy and diagrams to document the origin of TOS and their varied activities. However, the embodiments of the TOS may be in different forms and these figures should not be construed as limiting the scope of the utility of the TOS as described herein. The figures are illustrious of embodiments and are in accord therewith.

A novel biological entity with-biomedical utility in the form of tissue organizing structures, herein designated “TOS”, is described. The TOS were discovered as part of a newly identified process by which cancer cells organize in culture to form three-dimensional solid tumors. The tumor forming process involves these novel cell-derived structures that play a role in orchestrating the process of solid tumor formation and expansion. The studies document that TOS are produced by normal and malignant human cells, and also by cells of murine origin, suggesting that these novel structures are produced by cells of diverse mammalian species. Further studies document the discovery of TOS in species of primitive plants, suggesting that the evolutionary origins of these TOS date to early stages of metazoan evolution. The TOS are implicated in regulating biomedical processes associated with tissue organization and genetic recombination, tissue morphogenesis, malignant transformation, the phenotypic reversion of malignant transformed cells, and the specific induction of cell death in malignant tumor cells.

For a more complete understanding of the TOS and the advantages, reference is now made to the following non-limiting descriptions taken in conjunction with the accompanying examples, protocols and figures.

### Isolation, Purification and Identification of TOS

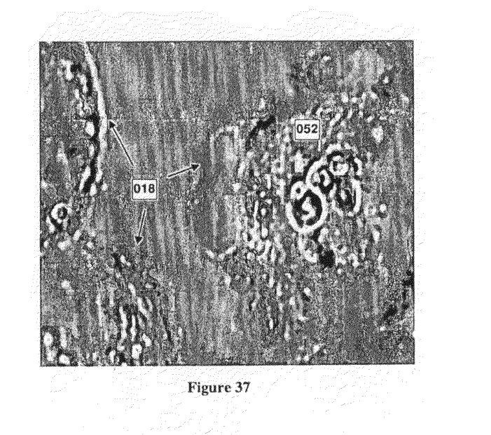

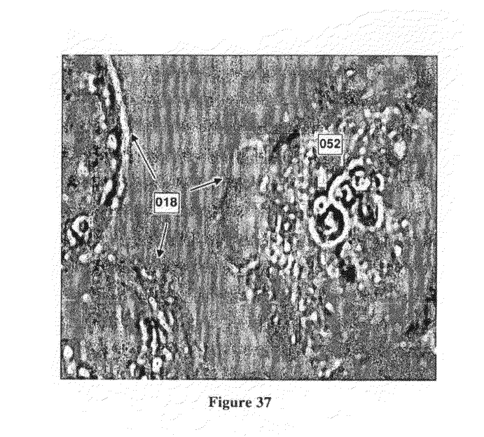

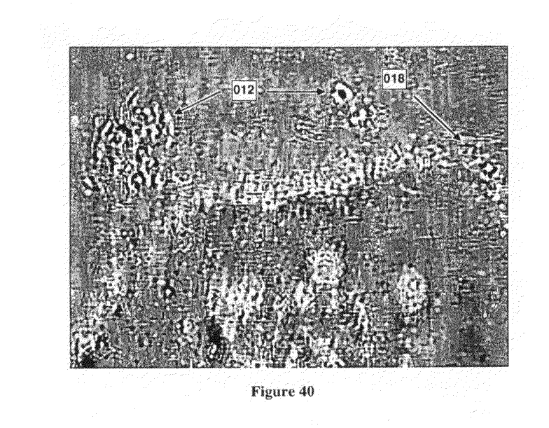

Referring now to the drawings, FIG. 1 is a diagram of an embodiment of TOS formation and tumor growth 010 wherein TOS 012 is induced in genetically altered cells 014, comparable to hyperplastic cells or emerging carcinoma in situ. As the cells 014 begin to abrogate their normal stromal attachments 016 in the tissue of origin, this initiates cell membrane changes to produce TOS “buds” 018 that induce the formation and release of TOS 012, a process that may be driven by a positive feedback loop mechanism 020, as TOS 012 give rise to more TOS 012 by subsequent ‘n’ rounds of fusion 022 with additional tumor cells 014 that also may trigger autonomously driven intracellular TOS replication 024 and reproduction of a cell into a plurality of TOS in the cell, or a TOS “bag” 026 as utilized herein, and then with rapid cell fission 028 TOS bodies 012 are released. Free TOS bodies 012 mediate cell-to-cell attachments 030 enabling tumor 010 growth. In an additional embodiment of tumor growth 032, free TOS bodies 012 aggregate to form TOS tubules 034 utilizing cellular ECM fibers 036. These tubules 034 then act as conduits and direct connectors to enable tumor 0320 growth. TOS 012 also appear to stimulate cell division in tumor cells 014 and somatic genetic recombinations that may contribute to genetic instability and morphogenetic changes occurring in tumors cells 014 with which they are associated. The formation of TOS tubules 034, their utilization of ECM components 036, their rapid mobility all taken together may orchestrate many of the dramatic cell movements that characterize the abnormal tissue organizations comprising solid tumor 010 and 032 structural components. Moreover, the TOS tubules 034 generate a circuitry of tumor cell origin that permits extensive tumor 032 spread and communication in the absence of angiogenesis. The activities of these unusual TOS bodies 012 account for many of the abnormal behaviors characteristic of solid tumor malignancy. In addition, FIG. 26, a frame from live cell videography, shows TOS-associated tubules 034 and cell extensions of GBM cells 052. FIG. 37, another frame from unstained live cell videography, shows TOS in various stages of budding 018 from a GBM cell 052 membrane. FIG. 40 is a frame from unstained live cell videography showing TOS budding and release from MDA-MB-231 breast carcinoma cell.

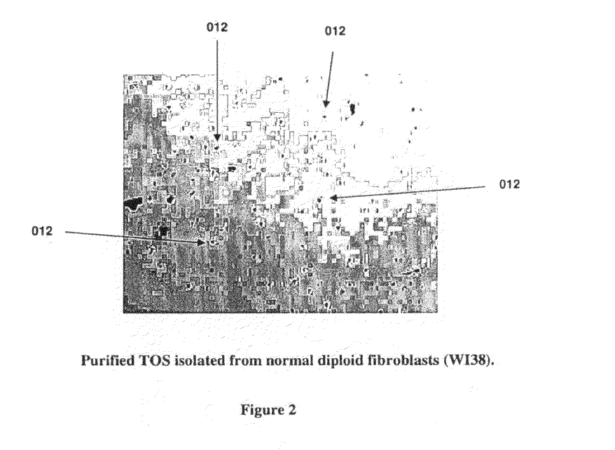

FIG. 2 is a photomicrograph (1000×) of an embodiment of TOS 012 isolated from normal diploid fibroblasts (WI38), the method of isolation comprising the steps of;

a. culturing W138 cells overnight in serum-free culture medium to stimulate the production of TOS in the cultured cells,

b. removing the culture media,

c. washing the cells with a composition of phosphate buffered saline,

d. treating the cells with a composition of trypsin for 30 minutes to facilitate cell accumulation of TOS,

e. adding 10% fetal bovine serum (FBS) to the trypsanized cells to stimulate release of TOS,

f. releasing TOS from the trypsanized cells by incubating overnight at 37 degrees centigrade wherein the released TOS bind tightly to the bottom of the culture dish,

g. removing the cells from the culture dish to isolate the bound TOS.

An embodiment of producing a purified composition of TOS 012 comprises the steps of,

a. culturing W138 cells overnight in serum-free culture medium to stimulate the production of TOS in the cultured cells,

b. removing the culture media,

c. washing the cells with phosphate buffered saline,

d. treating the cells with trypsin for 30 minutes to facilitate cell accumulation of TOS,

e. adding 10% fetal bovine serum (FBS) to the trypsanized cells to stimulate release of TOS,

f. releasing TOS from the trypsanized cells by incubating overnight at 37 degrees centigrade wherein the released TOS bind tightly to the bottom of the culture dish,

g. removing the cells from the culture dish to isolate the bound TOS,

h. removing the bound TOS from the culture dish by physical or other acceptable means to substantially maintain TOS activity, such as a cell scraper, and

i. resuspending the TOS in an amount of acceptable diluent to maintain TOS activity, such as culture medium, to provide a purified TOS composition.

The product is one hundred percent (100%) pure TOS as cells from which they were derived are destroyed by selective trypsinization. No other steps are required for purification. Other embodiments for TOS isolation and purification comprise high speed ultracentrifugation.

An embodiment for the study of tumor formation and isolation of TOS in vitro comprises utilizing tumor cells derived from diverse human cancer cell lines, including but not limited to cancers of the colon, lung, brain and breast, by plating the tumor cells following trypsinization on culture dishes coated with 1% agarose to block cell-to-substrate attachment in order to promote cell-to-cell interactions required for solid tumor formation. Videography or photomicroscopy imaging in real time was conducted of the live cell cultures, from the time of plating continuously for 4-5 hours post-plating and intermittently over a period of approximately one month. This procedure was utilized to record the overall chronology of early stage solid tumor development and to explore visually the behavior of individual cells as they begin the process of spontaneous associations that culminate in the formation of microscopic solid tumors in vitro. In addition, photomicroscopy and videography documentation of the tumor cell behavior was conducted to identify the TOS. For example, FIG. 37 is a frame from unstained live cell videography showing TOS in various stages of budding 018 from a GBM cell 052 membrane. FIG. 38 is a frame from unstained live cell videography showing TOS-mediated cell-to-cell interactions 031 (MDA-MB-231 breast carcinoma cells).

An additional embodiment of a method for isolating TOS comprises culturing cells in the absence of serum in pyruvate supplemented medium that generated conditions not conducive to sustaining cell viability while facilitating the culture and isolation of cell-free extracts of TOS for further characterization.

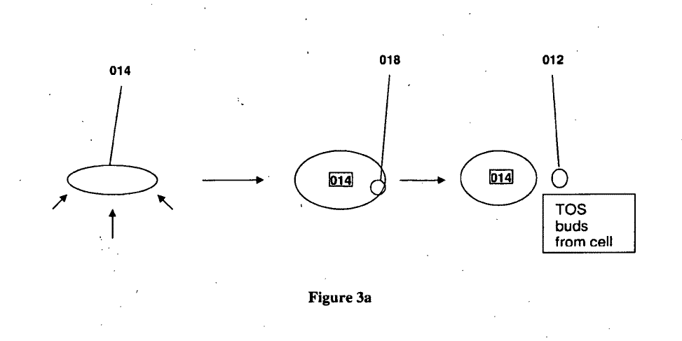

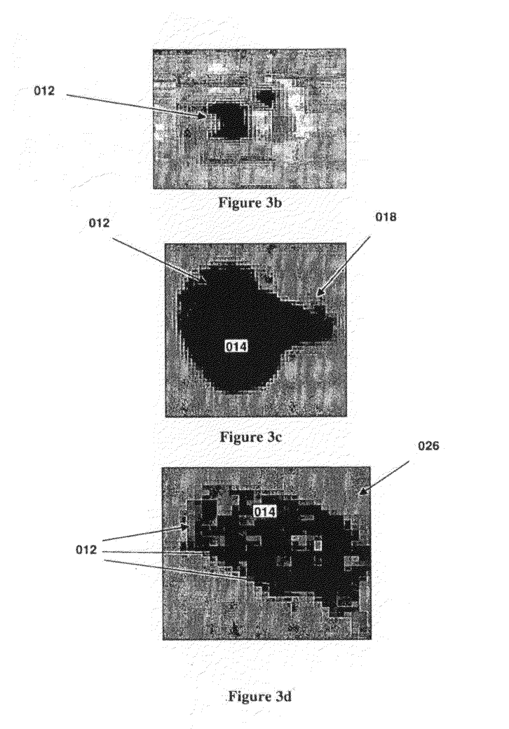

An embodiment of a method for identification of TOS comprises staining fresh tumor tissue with either hematoxylin/eosin, or periodic acid Schiff reagent/hematoxylin by known methods. Visualizing of the TOS by microscopy is employed with a record made by known photographic techniques. TOS isolated and stained according to the method described herein is identified in the photomicrograph of FIG. 3 at 1000× magnification having an approximate diameter of 2 microns in diameter.

TOS may be isolated from cells growing in culture by microscopic visualization of cells immediately following substrate detachment, an event that triggers TOS formation and budding from the cell surface. TOS may be visualized at high magnification (1000×) in live unstained cell preparations in culture and by staining (FIGS. 2, 3, 17, 22, 29, 30, 31, 35, 36 and 40). TOS morphology is spherical and the diameter is approximately 2 microns. Hematoxylin/eosin (H & E) staining reveals a densely staining interior of many, but not all, TOS particles, suggestive of nucleic acid content. Periodic acid/Schiff (PAS) staining of TOS particles stains the exterior surface and many extensions/tubules associated with TOS indicating the presence of lipid or lipids. The exterior surface is membrane like in its properties (budding, fusion) and its lipid-staining biochemical properties. The presence of nucleic acid in the interior of the TOS vesicles was confirmed using acridine orange staining and fluorescence microscopy, which indicated the presence of double-stranded nucleic acid in these particles. Rhodamine 1,2,3-staining produced a pronounced fluorescence indicative of energy production in these vesicles. Protein does not appear to be a major component of TOS particles as they are insensitive to trypsinization.

### TOS Assessment as Therapeutic Cancer Agent or Target

TOS possess unique properties associated with tissue organization and genetic recombination and have clinical utility in tissue and genetic engineering as well as in cancer prevention and therapeutic use and targeting. The studies of the earliest stages of solid tumor mass formation in vitro revealed that this process is critically linked to the formation of TOS bodies. These previously unidentified structures appear to play a central role in many aspects of tumor cell behavior associated with solid tumor development in vitro.

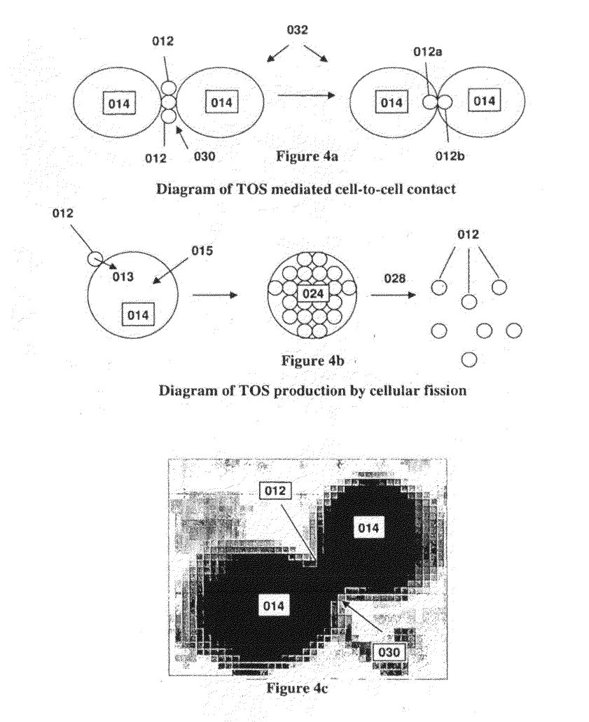

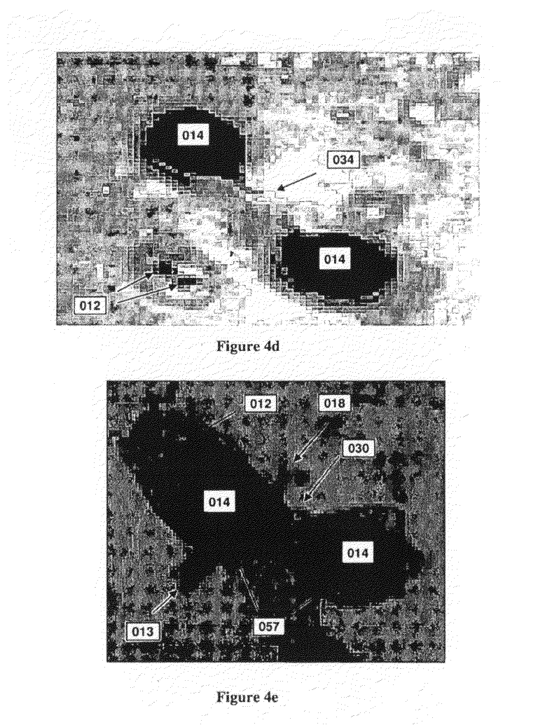

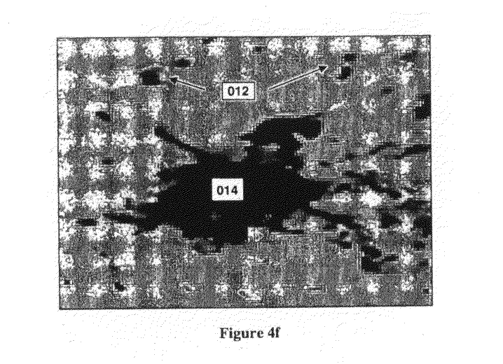

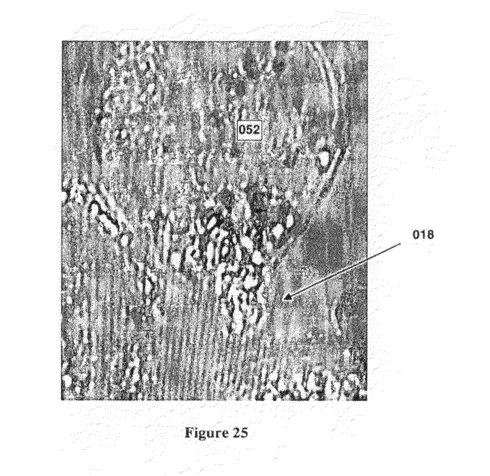

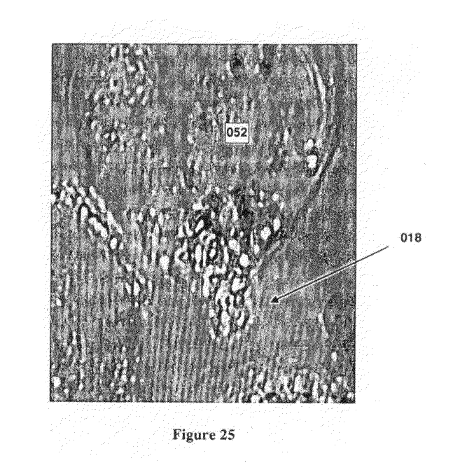

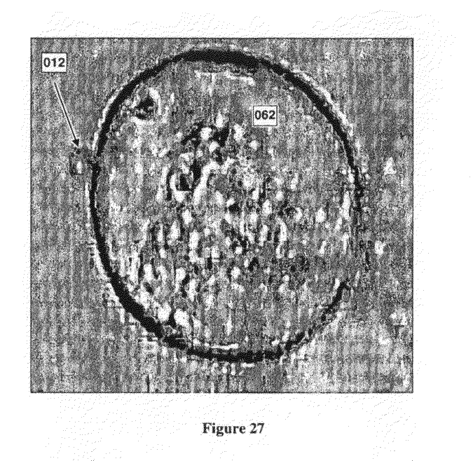

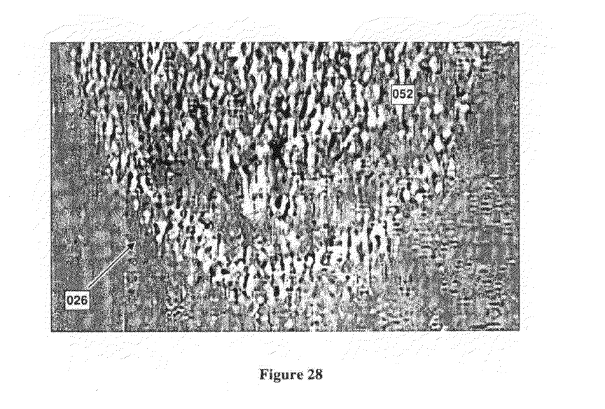

In the embodiment of FIG. 3a, a model of TOS production by budding is presented wherein a TOS particle 012 “buds” 018 from a tumor cell 014. FIG. 3b is a photomicrograph (1000×) of an individual TOS body 012 isolated from hematoxylin/eosin stained, glioblastoma multiforme DBTRG.05MG cells, having an approximate size of 2 microns. A single particle is shown with a densely stained central area. In FIG. 3c, the spatio-dynamic data that was obtained from microscopic observations of live tumor imaging and H & E staining for structural assessment indicated the TOS bodies 012 may form as “buds” 018 on the surface membrane of tumor cells 014 and are then activated by substrate/stromal detachment 016. The image shows the morphogenesis of tumor cell (GBM) to TOS—producing cell. Note the interior of densely staining TOS aggregates. FIG. 3d is a photomicrograph (1000×) of a tumor cell 014 showing TOS bodies 012 formed via a fission-like process (also see FIG. 4a) in which a tumor cell 014 is induced to undergo a rapid morphogenesis and then lyse to release a plurality of TOS 012. The H & E stained cell preparation shows complete conversion of the tumor cell (GBM) to a “TOS bag”—a cell consisting of emergent TOS particles to be released by cell fission. The densely stained interior particles are associated with TOS formation. In addition, FIG. 25, a frame from live cell videography, shows TOS budding 018 from the surface of glioblastoma GBM cells 052. FIG. 27, another frame from live cell videography, shows TOS 012 attaching to NCI-H1299 lung carcinoma cells 062. FIG. 28, another frame from live cell videography, shows TOS-induced morphological transformation of GBM cell 052 to “TOS bag” 026.

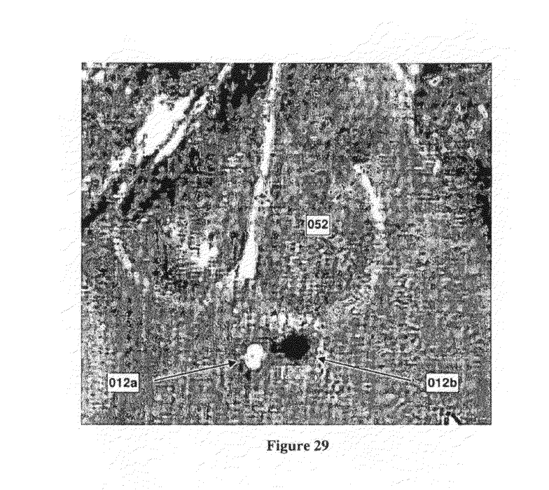

TOS 012 in FIG. 3 appear to be membrane-delimited vesicles that frequently contain genetic material based on their staining properties, although empty vesicles that do not contain genetic material have also been observed. FIG. 29, a frame from live cell videography shows empty TOS 012a (light) interacting with dense TOS 012b (dark) and cell membranes at the border of solid tumor GBM cells 052. Other properties of TOS include rapid mobility and the capacity to fuse with other TOS 012 and recombine genetic material before separating, an event that is sometimes associated with cell divisions in duplicate or triplicate. Alternatively, TOS 012 may enter tumor cells 014 by a process that appears to occur by membrane fusion 022 and then induce rapid morphological changes in the host tumor cell 014, including morphogenesis to “TOS bags”, which is a cell 014 containing a plurality of TOS 012 prior to lysing and release.

TOS 012 were observed to spread rapidly throughout the culture by several means, including the use of extra-cellular matrix (ECM) fibers 036 produced by the tumor cells 014 as pathways from one cell mass 032 to another. In addition, TOS 012 were capable of organizing into tubular masses following their release from tumor cells to form TOS tubules 034 as connectors linking various areas of the tumor mass 032, as shown in FIG. 1.

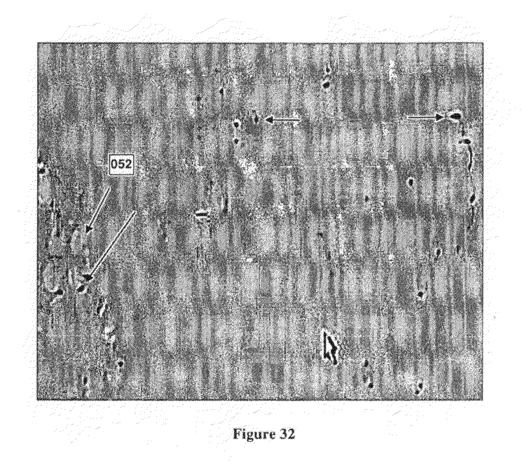

FIGS. 4a and 4b are embodiments of two diagrams depicting TOS mediated cell contact and TOS produced by cellular fission. In FIG. 4a, solid tumor formation 032 is indicated generally showing that TOS bodies 012 and/or tubules 034 formed from TOS 012 mediate cell 014 to cell 014 contact 030 to produce cells conjoined by TOS bodies 012a and 012b. This process is repeated thus allowing TOS 012 to act as the mediator for tumor 032 growth. FIG. 4b illustrates generally TOS 012 production by cellular fission 028, where cell 014 is contacted by TOS body 012 which infuses TOS material 013 into the cell 014 cytoplasm 015, TOS bodies 012 then replicate 024 within the cell 014 and are released with cell fission or lysis 028. FIG. 4c is a photomicrograph (1000×) of NCI-HI299 lung carcinoma, hematoxylin/eosin stained cells 014, at day one of solid tumor 032 formation showing TOS 012 mediated cell fusion in cell-to-cell contacts 030 associated with TOS 012 in accord with the illustration above. FIG. 4d is a photomicrograph (1000×) of H & E stained GBM tumor cells 014, illustrating that TOS 012 mediated cell fusion may progress to elaborate tubular structures, TOS tubules 034, between cells. Fused TOS particles 012 with densely staining interiors interacting may indicate genetic recombination. FIG. 4e is a photomicrograph (1000×) of DBTRG.05MG glioblastoma multiforme, periodic acid/Schiff reagent stained cells, showing cell-to-cell contact 030. The fused tumor cells 014 display internal TOS 012 formation, individual TOS budding 018, and a larger aggregate 013 budding from cell 014. Note the enlarged nuclei 057. FIG. 4f is a photomicrograph (1000×) of an H & E stained GBM tumor cell 014 showing fission of the TOS filled cell and release of TOS particles 012. The net result of these TOS activities appeared to be directly associated with cell-to-cell associations and the formation of intercellular structures to facilitate the association and spread of tumor masses within the culture vessel. In FIG. 32, a frame from unstained live cell videography, early stage solid tumor formation of GBM cells 052 in culture is shown. Note the arrows indicating dense areas concentrated at sites of cell polarization and migration. These often serve as initiation sites for TOS formation.

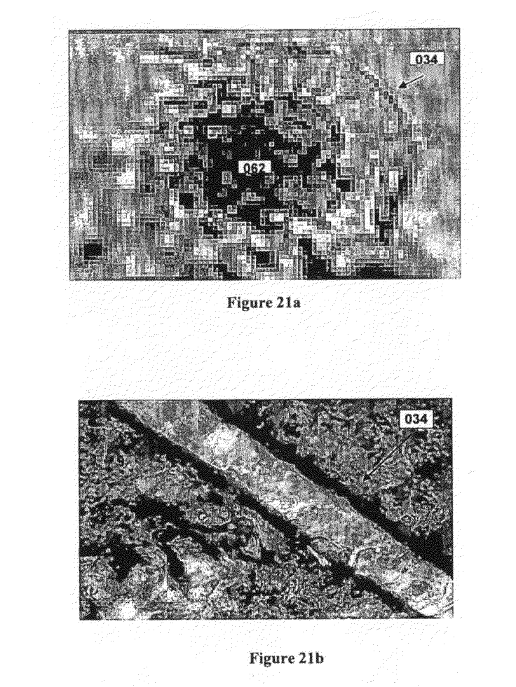

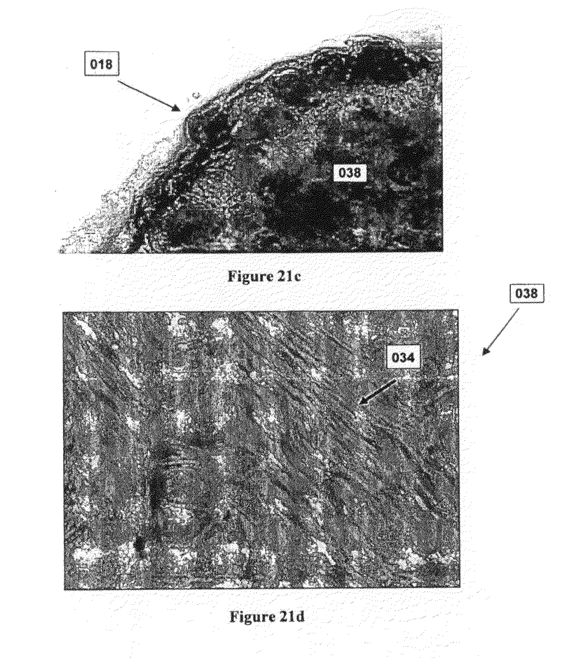

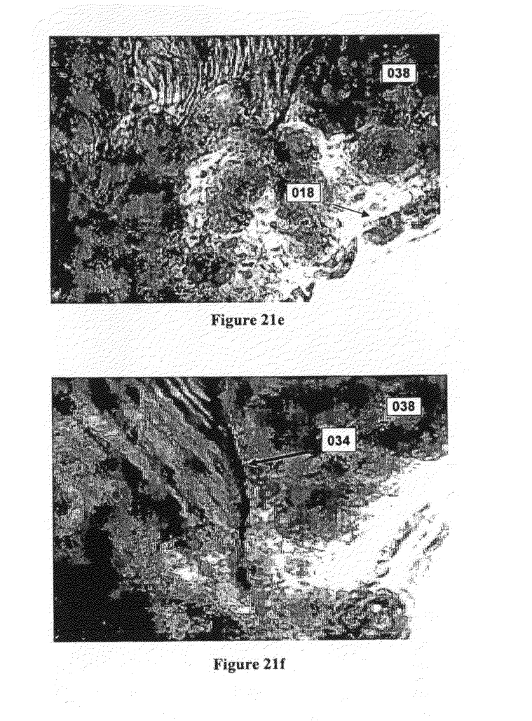

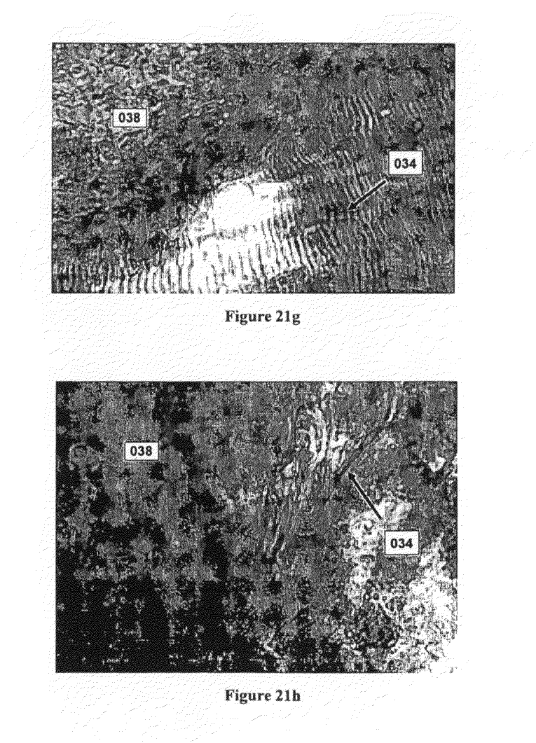

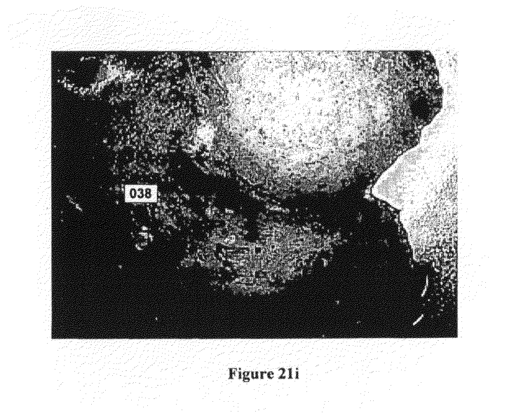

Based on the live microscopy studies of the spatio-temporal behavior of tumor cells at early stages of solid tumor formation in vitro, it appears that TOS are plasma membrane delimited cell-derived structures that conduct a central role in orchestrating the movements and cell-to-cell interactions of tumor cells essential to the formation of microscopic solid tumors, and is the presumable initiation complex fundamental to the establishment of solid tumor malignancies. In FIG. 21, the observed TOS-tumor cell interactions and tubular extensions of solid tumor masses resulting from the TOS interactions suggest that these novel structures may play a critical role in processes associated with tumor spread and metastasis, both fundamental components of the malignant phenotype.

As shown in FIGS. 3 and 4, TOS exert a dramatic capacity to effect structural and functional alterations in tumors with which they associate almost immediately upon entry into the tumor cell, a process that appears to occur by cell fusion. This effect is clearly manifest in changes in the appearance of the cell surface, the movements of the cell induced by this association and also by the apparent interaction of these vesicles with the genetic material of the tumor cell, both as a mechanism by which they may acquire their genetic contents and also by altering the genetic contents of the tumor cell. These TOS-tumor cell associations also appear to serve as a stimulus for tumor cell fission to form “TOS bags” that subsequently release with cell lysis a plurality of TOS bodies that spread throughout at least the tumor area. This fission process may represent a novel form of autonomous replication process.

### Malignant Cell Induction or Transformation by TOS

Tissue organizing structures (TOS) derived from human malignant tumor cells are capable of inducing the morphological and biological phenotypic transformation of normal diploid fibroblast cells to a highly aggressive, malignant transformed cell line that was established as a stable immortalized cell line in vitro for several months.

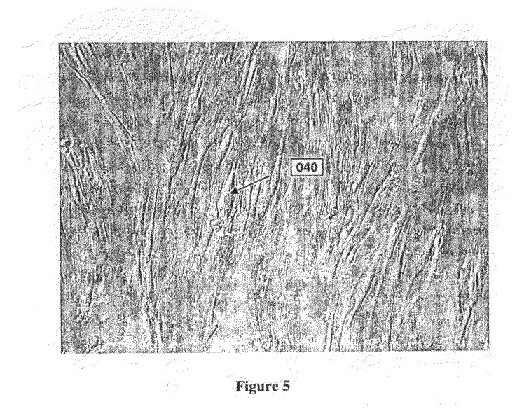

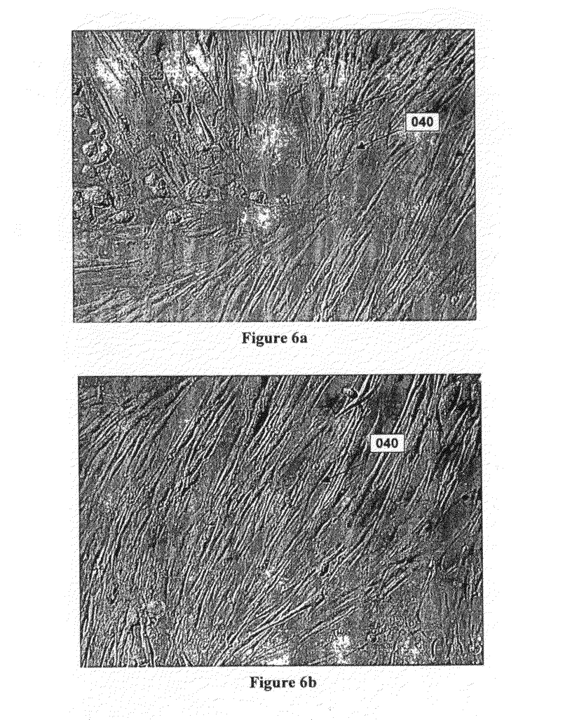

FIG. 5 is a photomicrograph (1000×) of a live cell preparation of normal W138 lung diploid fibroblasts 040 prior to exposure of TOS control. FIGS. 6a and 6b are photomicrographs (1000×) of live cell preparations of normal diploid lung fibroblasts (WI38) 040 after exposure to TOS control 044 (not shown) derived from W138 normal diploid fibroblasts 040. The figures demonstrate untreated control versus cells treated with normal TOS, which produced no demonstrable morphological effect.

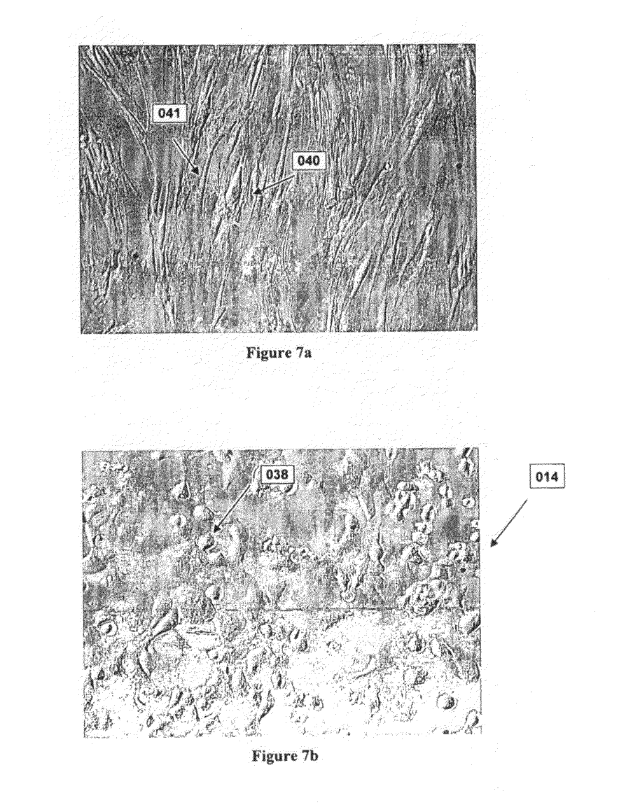

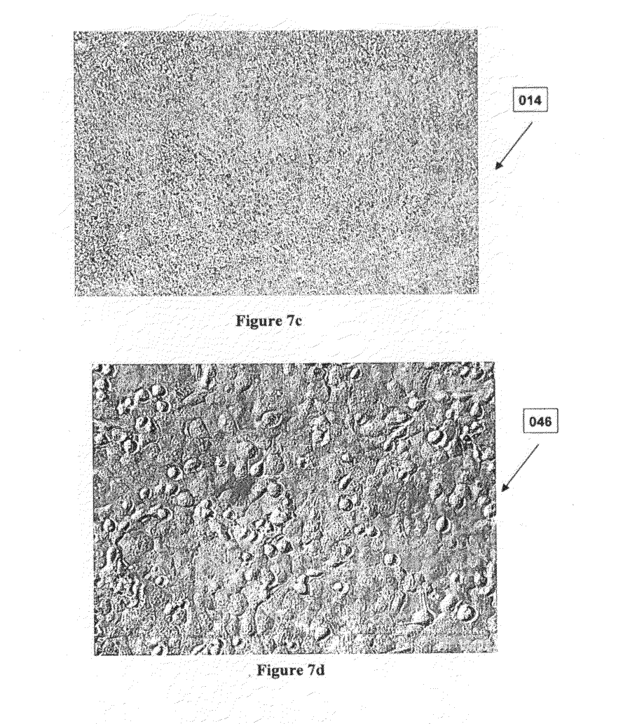

FIGS. 7a-7d are photomicrographs (1000×) of unstained live cell preparations of normal diploid lung fibroblast (WI38) cells 040 in monolayer culture. FIG. 7a shows normal lung diploid fibroblasts 040 prior to TOS 042 exposure and transformation. The elongated fibroblastic cell extension 041 is a characteristic morphology of this cell line. In FIG. 7b the normal cells 040 have been exposed to TOS 042 (not shown) derived from NCI-HI299 non-small cell lung carcinoma. Note the dramatic change in normal cell 040 morphology where the cells 040 have lost their characteristic fibroblastic extensions 041 thus showing malignant transformation of the normal lung fibroblasts 040. Within several days the 040 cells began to assume a rounded-up, spheroid 038 morphology and developed biological properties of malignant transformed cells 014. FIG. 7c is an unstained live cell photograph showing the TOS transformed fibroblasts 014 after 10 days resulting in a confluent monolayer of TOS-transformed lung fibroblasts. FIG. 7d is a photomicrograph of live cells showing the TOS transformed cell line 046 after several weeks in culture. The TOS 042 mediated morphogenetic transformation generated a stable phenotypic alteration. The morphogenetic properties of these cells 046 were distinct from the normal diploid fibroblasts 040 from which they were derived. They are similar in appearance, but display aggressive growth properties, unlike the finite normal cell line 040 from which they originated, similar to the lung carcinoma cells used to prepare TOS 042 for the transformation.

The methodology to transform the normal cells into malignant cells having the phenotype of the source malignant cells involved the application of purified TOS 042 prepared from the NCI-HI299 non-small cell lung carcinoma malignant cells to a monolayer culture of normal lung fibroblasts 040, comprising the steps of:

a. isolating and purifying TOS from NCI-H1299 lung carcinoma cell using the procedure described above,

b. suspending TOS in phosphate buffered saline solution isolated from approximately 2×10̂5 cells,

c. adding the TOS suspension to a culture dish containing a monolayer of normal WI38 lung cells (approximately 1×10̂5 cells) growing in culture medium,

d. incubating the TOS-treated cells at 37 C in 5% CO2, and

e. producing TOS transformed malignant cells comprising the phenotype of the source malignant cells.

The TOS 042 and cell 040 interactions resulted in the morphological transformation of the normal lung fibroblasts 040 to cells 046 that distinctly resembled the lung carcinoma cells from which the TOS 042 were derived. Morphological transformation occurred over a period of several days to one week and produced a stable cell line 046 displaying many properties of transformed cells 014, including: immortalized growth, rapid mitosis, anchorage independence and ability to form solid tumors in vitro. These malignantly transformed cell lines 046 were maintained in culture for a period of more than three months without any loss of proliferative capacity, thus behaving as a continuous cell line, distinguishing them from the finite fixed lifespan cell line 040 from which they were derived.

These cell lines 046 have been maintained as a genetically stable novel cell line 046 in vitro capable of anchorage-independent growth and these cells form solid tumor spheroids 038. In contrast, the pre-transformed normal W138 cells 040 require substrate attachment characteristic of anchorage-dependent growth and viability, and are incapable of generating solid tumor spheroids 038, a characteristic of malignant cells. Thus the TOS-transformed cell line 046 displays widely divergent properties distinguishing these transformed cells 014 from the normal lung fibroblasts 040 from which they were derived. Moreover, the TOS-transformed cells 046 display growth properties in vitro associated with a higher proliferative rate than the lung carcinoma cells from which the TOS were derived, consistent with a highly malignant transformed state.

This data suggests a novel mechanism of malignant transformation associated with TOS activity, and has increased the scope of functional activities associated with these biological entities. This mechanism has important potential clinical applications defining TOS as a therapeutic target based on the observed transforming behaviors of tumor-derived TOS.

The observed phenotypic transformation suggests that TOS are capable of transferring genetic material to host cells with which they are associated in a stable fashion which may be associated with genomic integration of exogenous TOS-associated DNA. The DNA transfer function of TOS is further indicated by additional studies demonstrating that TOS derived from green fluorescent protein(GFP)-transformed mouse endothelial cells are cable of inducing the phenotypic transformation of human glioblastoma multiforme (GBM) tumor cells such that they acquire a GFP+ fluorescent phenotype following exposure to TOS derived from GFP+ cells.

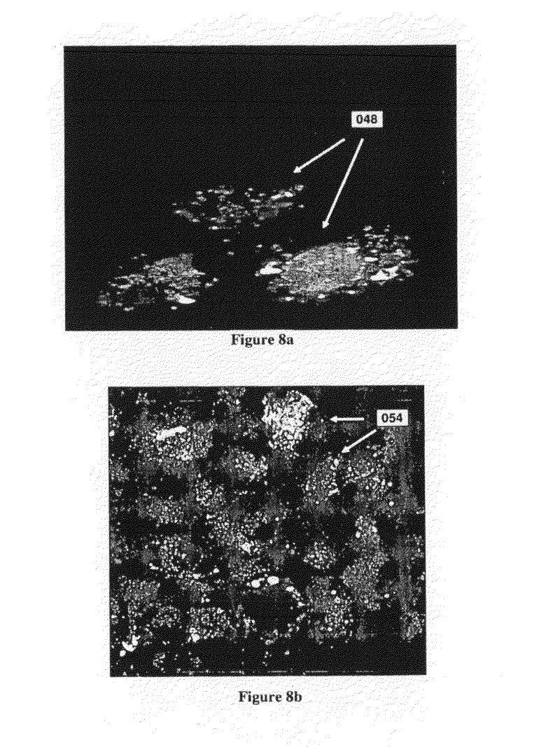

In FIGS. 8a and 8b (Confocal laser fluorescent microscopy I000×), TOS 048 prepared from mouse endothelial cells that contain the GFP (green fluorescent protein) gene 050 transformed human glioblastoma multiforme cells (GBM) 052 to a GFP+ fluorescent phenotype cell 054 by the method comprising the steps of,

a. isolating and purifying TOS from mouse endothelia cells that contain the GFP gene using the procedure described above,

b. suspending the TOS in phosphate buffered saline solution,

c. adding the TOS suspension to a culture dish containing a monolayer of human glioblastoma multiforme cells (GBM) growing in culture medium,

d. incubating the TOS-treated cells at 37 C in 5% CO2, and

e. producing TOS transformed cells comprising the GFP+ phenotype of the source cells.

Additional studies documented the presence of DNA 056 in TOS by acridine orange staining and fluorescent photomicroscopy by methods known in the art, comprising

a. isolating and purifying TOS from cells in culture by the methods described herein,

b. staining the TOS particles with acridine orange fluorescent stain according to standard established procedures,

c. and examining the stained TOS particles by fluorescent microscopy.

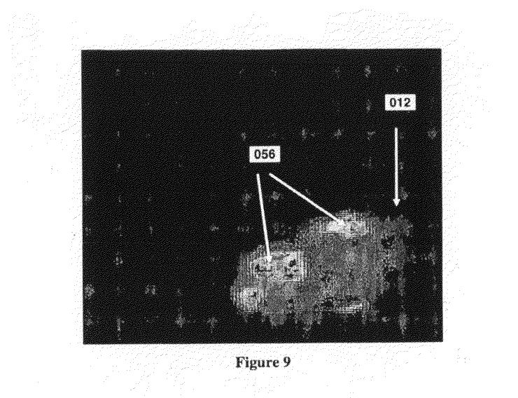

In FIG. 9, TOS particles 012 stained with acridine orange indicate the presence of double-stranded DNA 056 by confocal fluorescent laser microscopy (1000×).

These TOS-transformation studies suggest that the source of the DNA 056 component of the TOS 012 is the host cell genomic DNA as the phenotypic properties of TOS-transformed cells 014 vary depending on the cell source of the TOS 012. These studies further suggest that TOS 012 are capable of engaging in genetic recombination between DNA 056 sequences carried in the TOS 012 and host cell genomic DNA. The recombinational properties of. TOS 012 may comprise the basis of their ability to induce the morphogenetic transformation of cells with which they interact. These recombinational properties have profound applications for novel gene therapy approaches to the treatment of many diverse human diseases.

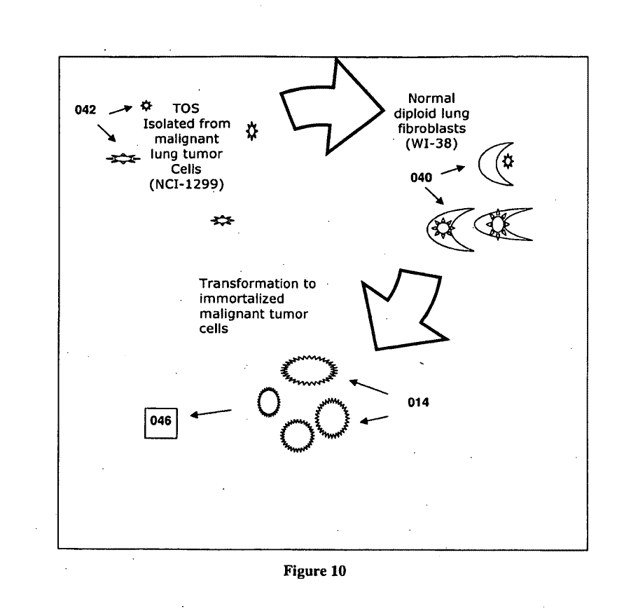

FIG. 10 is a diagram of an embodiment of a method of a TOS cellular transformation process. TOS 012 extracted and purified from malignant lung carcinoma cells 042 are added to cultures of normal diploid fibroblasts 040. Within several days, the normal cells 040 begin to assume the properties of transformed malignant cells 014 and can be established as a permanent cell line 046.

### Malignant Cell Reversion by TOS

Tissue organizing structures (TOS) 044 derived from normal diploid fibroblasts 040 are capable of inducing the morphological and biological phenotypic reversion of the malignant cell line 046 established by TOS transformation, ultimately inducing transformed malignant cell death.

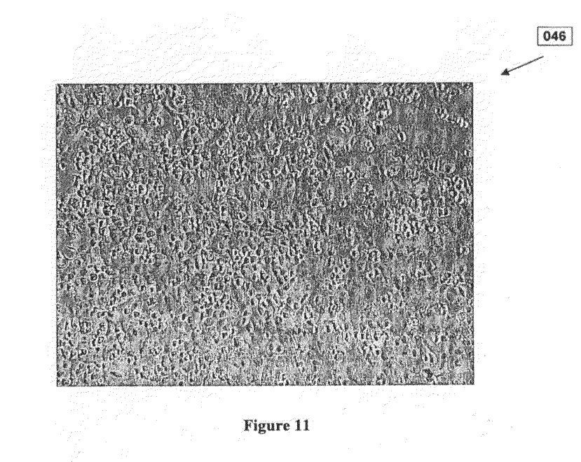

FIG. 11 is a photomicrograph (1000×) of an unstained live cell preparation of TOS transformed lung fibroblasts 046 as the control wherein no normal TOS has yet been added, and thus acts as a negative control.

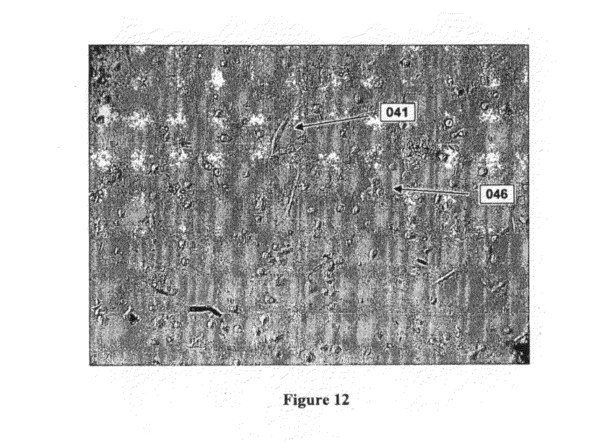

FIG. 12 is a photomicrograph (1000×) of an unstained live cell culture of TOS transformed tumorigenic lung cells 046 exposed to normal TOS 044 (not shown) after 24 hours. TOS 044 induce a partial phenotypic reversion of the lung carcinoma cells 046 as cell extensions 041 characteristic of normal fibroblasts 040 are identified. Thus, the photo shows evidence of morphogenetic reversion of TOS-transformed cells to a more fibroblastic appearance and evidence of cytotoxicity.

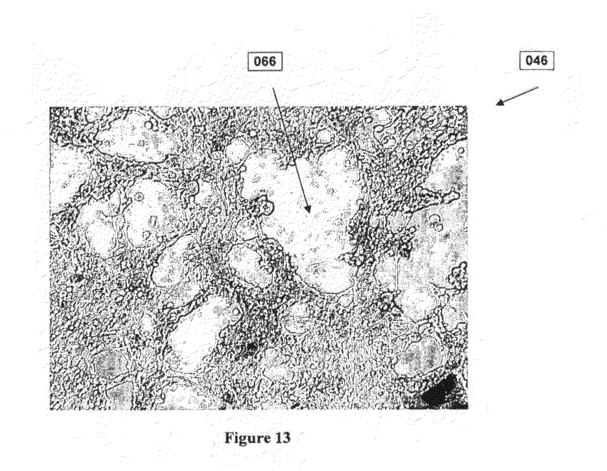

FIG. 13 is a photomicrograph (1000×) of an unstained live cell culture of TOS transformed lung fibroblasts 046 exposed to normal lung TOS 044 after ten days. At ten days, the TOS-treated malignant cells are growing poorly with open areas appearing as syncytial plaques 066. Thus, the photo shows evidence of massive cell death of transformed cells, suggesting that TOS from normal cells may have a therapeutic destructive effect on abnormal malignantly transformed cells in culture.

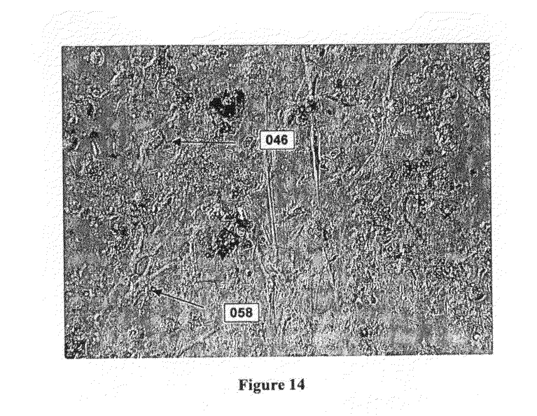

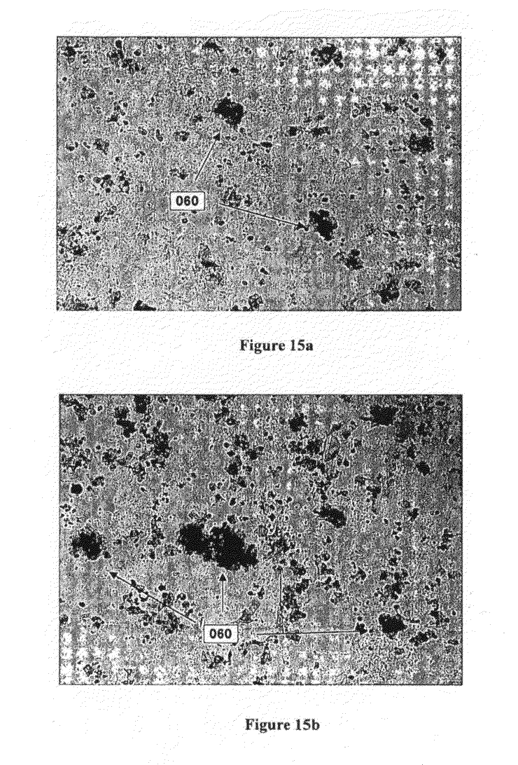

FIG. 14 is a photomicrograph (1000×) of an unstained live cell culture of TOS transformed lung fibroblasts 046 exposed to 2× normal TOS 044 24 hours. Phenotypic cell reversion 058 is concentration dependent as pronounced morphological effects are observed when 2× purified TOS is applied to malignant cells 046 growing in culture. 058 indicates a morphologically transformed cell. Thus, the photo shows concentration-dependent effects of therapeutic TOS activities

FIG. 15 is a photomicrograph (1000×) of an unstained live cell preparation of TOS transformed lung fibroblasts 046 exposed to 2× normal TOS 044 after ten days. The increased concentration has produced clusters of dead cells 060. Compare to the cell death in FIG. 13.

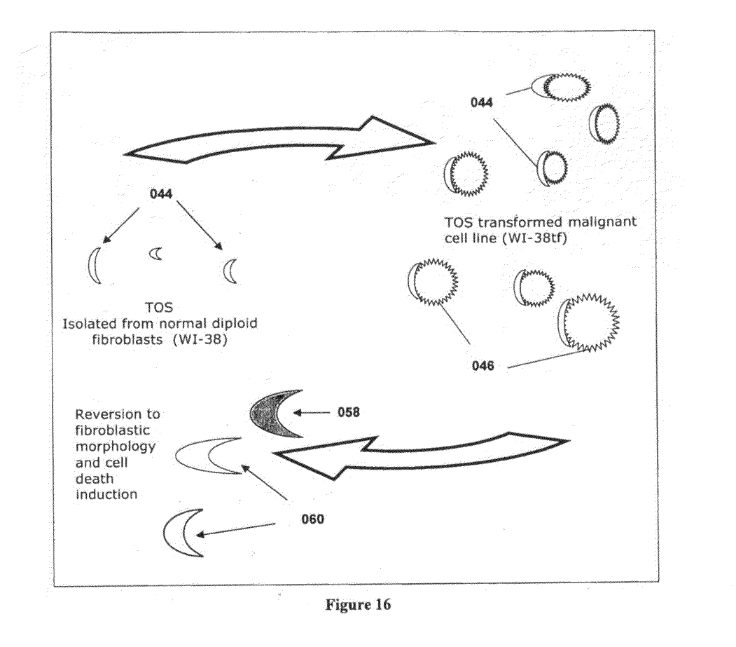

FIG. 16 is a diagram of an embodiment of cellular reversion by TOS. TOS 044 derived from the normal lung fibroblast cell line (WI38) 040 were observed to induce the phenotypic reversion 058 of previously TOS transformed lung cells 046 such that they assumed a normal fibroblastic phenotype 040 which preceded the induction of cell death 060 in the TOS-transformed cells 046. Procedurally, TOS 044 extracted and purified from normal WI-38 human diploid fibroblasts 040 were added to cultures of TOS-transformed malignant cell line 046. Within several days phenotypic reversion 058 and cell death 060 occurred in the transformed cell line 046. The rate of phenotypic reversion 058 and degree of cell death induction 060 were correlated with the amount of purified TOS 044 added to the cultured transformed cells 046. Although quantitative methods of TOS assessment are currently still in development, it was estimated that TOS 044 purified from approximately 1×10>4 cells 040 were capable of inducing the complete cell death 060 of 1×10>6 cells 046 per mL in culture. This observed phenotypic reversion 058 of a malignantly transformed cell line 046 by TOS 044 derived from normal cells 040 suggests that TOS 044 may incorporate functions that have significant potential therapeutic value. In addition, it was observed that the TOS 044 derived from normal lung cells 040 induced the death 060 of the phenotypically reverted transformed cells 046, while displaying no discernible morphogenetic or toxic effects when combined with the normal lung cells 040 from which they were isolated. These data provide further evidence that TOS 012 derived from different cell and tissue types contain unique DNA 056 components specific to their tissue of origin and are capable of effecting morphogenetic alterations of unrelated tissues, including the reversion 058 of a disease-associated phenotype.

TOS derived from normal tissue can be utilized as therapeutic agents in the treatment of cancer based on studies demonstrating that TOS from normal cells are capable of inducing phenotypic reversion of transformed tumor cells in vitro associated with the induction of cell death.

TOS Isolated from Primitive Plant Species

TOS derived from plant tissue can be utilized as therapeutic agents in the treatment of cancer based on their ability to elicit selective cytotoxic responses in human malignancies.

In addition, TOS were identified and purified from a species of fern, indicating that these novel cell entities are ubiquitously present in widely divergent phyla, suggesting a common evolutionary origin that may be associated with the beginnings of the metazoan system. This notion is consistent with the apparent tissue organizing regulatory activities of these biological entities which may implicate these sub-cellular structures in embryogenic and developmental pathways essential to the formation of the multi-cellular system.

An embodiment of plant TOS extraction and purification procedure comprises: Plant material (leaves and stems) are washed in alcohol, then rinsed with sterile water. Cell culture media containing 10% FBS is added to the cleaned plant material. The mixture is then homogenized for approximately 3 minutes. The homogenate is then cultured at 37 C for 24-72 hours. The media/homogenate is removed leaving the plant TOS adhered to the culture flask. Plant TOS are scraped from the flask with a cell scraper in pure form in the absence of plant cells or other materials.

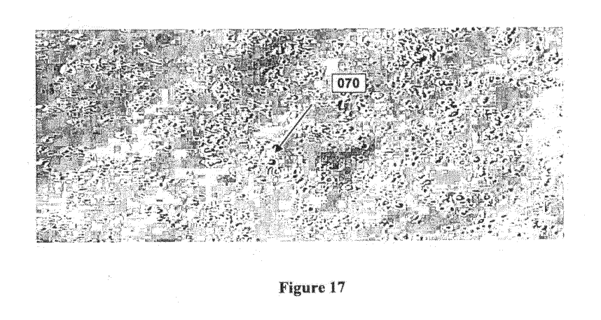

FIG. 17 is a live cell photomicrograph (1000×) of normal TOS 070 extracted from primitive plant (fern).

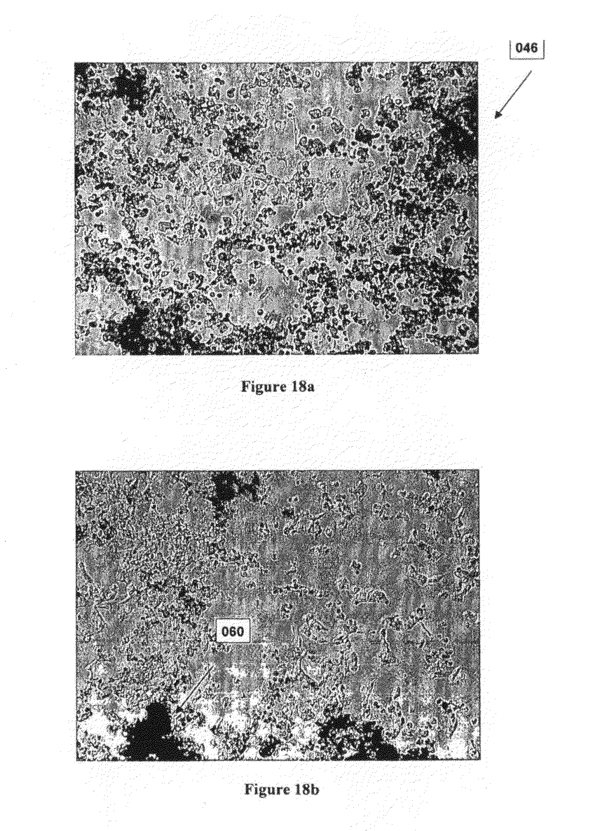

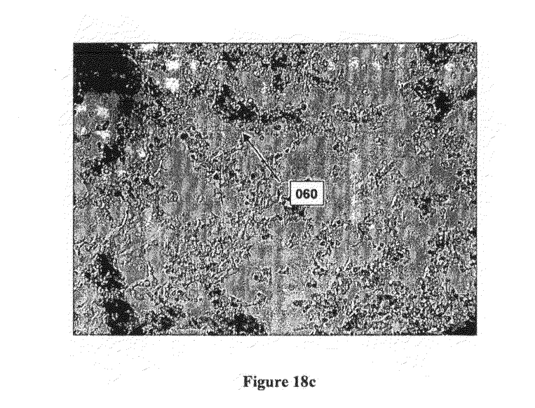

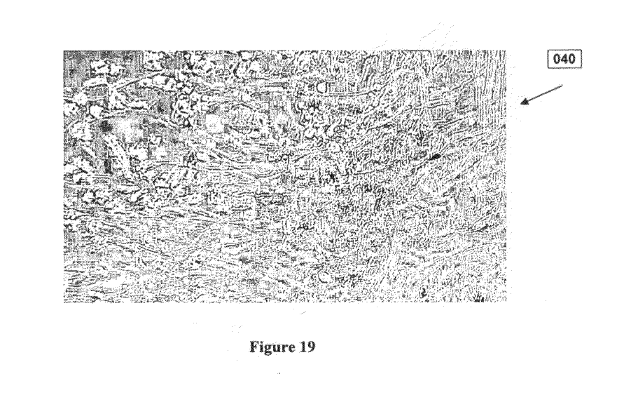

In FIGS. 18 and 19, TOS isolated from the fern species were found to display important cytotoxic activities specifically targeting malignant cancer cells while sparing normal cells in culture. FIGS. 18a-18c are photomicrographs (1000×) of unstained live cell preparations of TOS transformed lung fibroblasts 046 exposed to plant TOS 070 (not shown) at approximately two weeks. Note the clusters of dead cells 060 as in FIG. 15. Compare the result to FIG. 19, a photomicrograph (1000×) of unstained live cell preparations of normal diploid fibroblasts 040 exposed to plant TOS 070 (not shown) at approximately two weeks. The experimental procedure for FIGS. 18 and 19 comprised applying plant TOS 070 in purified form to cultures of NCI-HI22 non-small cell lung carcinoma 062 or WI38 normal diploid lung fibroblasts 040. The cells 040 and 062 were incubated in culture medium contain plant TOS 070 for approximately five days. The plant derived TOS 070 induced extensive cell death 060 in the malignant lung cancer cells 062 while displaying no significant cytotoxic effects in the normal lung cells 040 incubated under identical conditions. This observation suggests an important potential clinical therapeutic activity for the plant derived TOS 070.

The documented tissue transforming properties of TOS have important therapeutic application in the treatment of many other diseases associated with the loss or absence of normal tissue-associated functions, including recessive genetic disorders resulting from single gene mutations such as enzymopathies, relatively common disorders such as cystic fibrosis and muscular dystrophy, and loss of function related to diseases such as diabetes or tissue injury or damage. Treatment of these disorders by exposing the diseased tissues to TOS derived from normal cells of the same tissue type might be expected to induce the morphological and functional reversion of these tissues to a normal tissue type.

### TOS and Clinical Application for Tissue Engineering

Observation indicated that these unique cell-associated structures are highly pleomorphic with respect to their structural parameters and have extraordinarily diverse functionally with respect to their ability to interact with each other and with tumor tissue. FIG. 4 above demonstrates that cell-to-cell attachments are mediated by TOS interactions. High levels of TOS activity and concentration were associated with points of cellular interaction that ultimately became intercellular connections or junctions. The association of TOS with extra-cellular matrix (ECM) fiber structures appeared to tether the TOS to their contact cells to direct their movements in organizing the cell-to-cell associations characteristic of this primitive and fundamental component of solid tumor interactions. Direct entry of the TOS into cells, by what appeared to be membrane fusion followed by re-emergence, was observed to be associated with the activation of the membrane activity and motility of these cells, thus propelling them to interact with neighboring cells to form distinct cell-to-cell contacts.

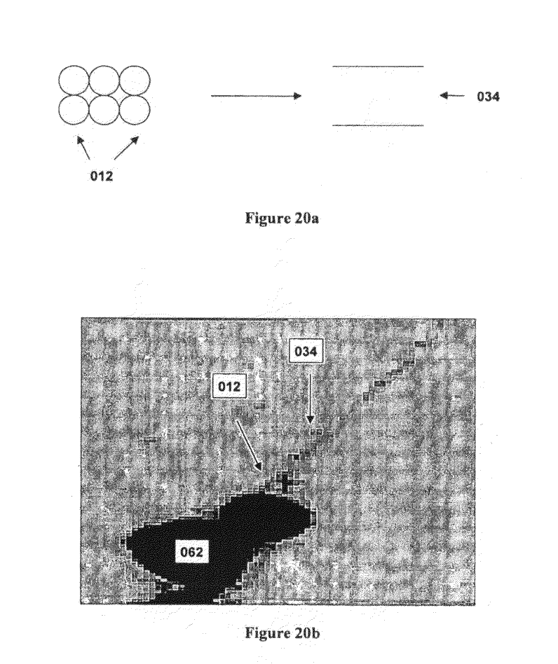

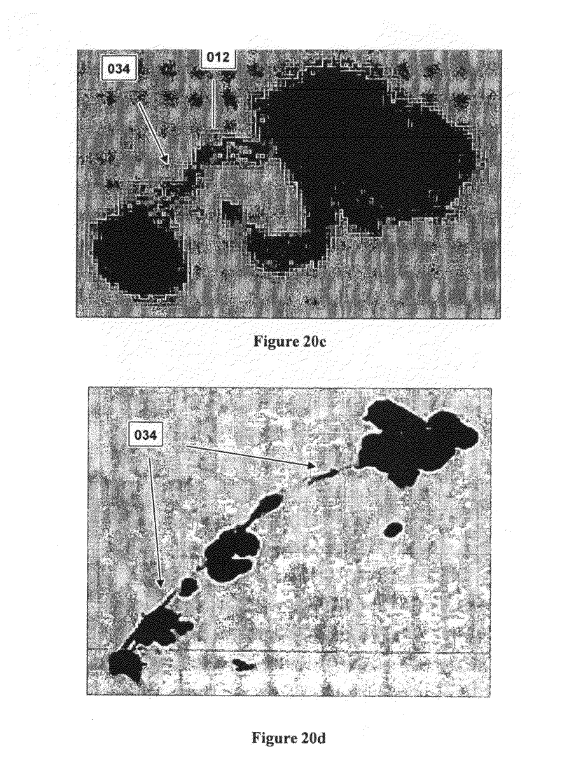

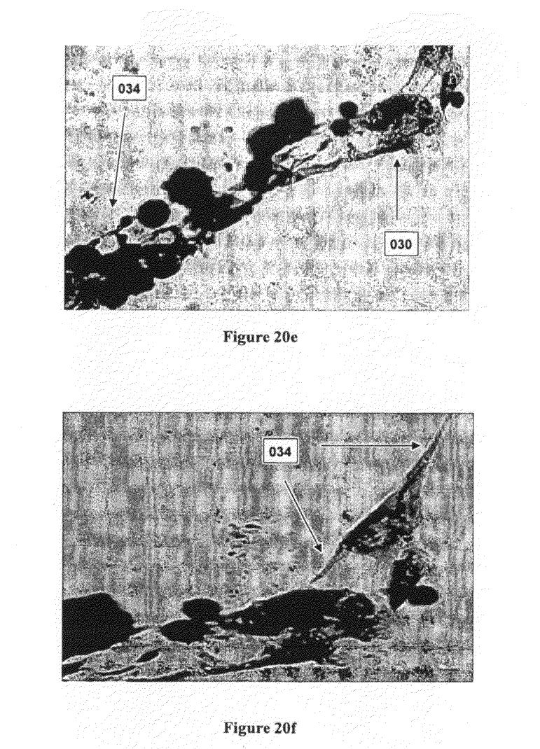

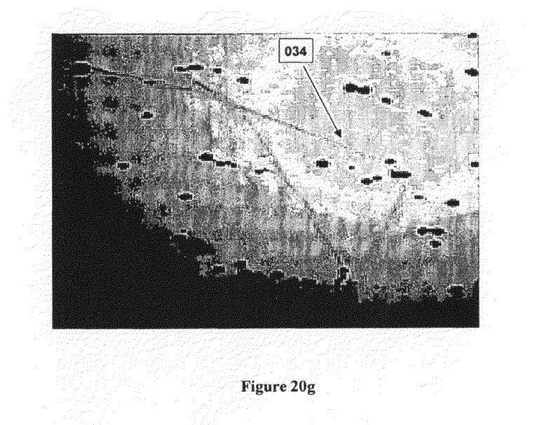

As shown in FIGS. 20a-g, the motility of these cells containing TOS 012 propelled them to interact with neighboring cells by utilizing TOS tubules 034 to form distinct cell-to-cell contacts 030. FIG. 20a is a diagram of an embodiment illustrating formation of TOS tubules 034 from fusion of individual TOS 012. In FIGS. 20b-20g, non-small cell carcinoma NCI-H1299 062 was stained with periodic acid/Schiff reagent and photomicrographed (1000×) at day 1 of solid tumor formation. FIG. 20b shows TOS tubule 034 formation by TOS 012 fusion from a small tumor cell cluster 064. Note the TOS 012 emerging from the cell 062. FIG. 20c shows early stage tubule 034 formation and TOS bodies 012 clearly evident. FIGS. 20d-20f show TOS tubules 034 joining small clusters of tumor cells 064. FIG. 20d shows tubules 034 formed from fusing TOS 012 to facilitate interactions between diverse clusters of cells in culture. FIG. 20e shows TOS tubule 034 interface between cell and TOS tubule 034 boundary, and TOS mediated cell connections 030. FIG. 20f shows TOS tubule 034 formation. FIG. 20g shows an overview of tubules 034 joining distant regions of tumor 064 at early stage formation 032.

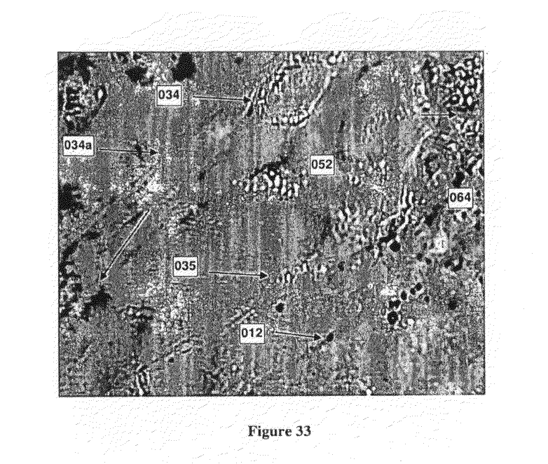

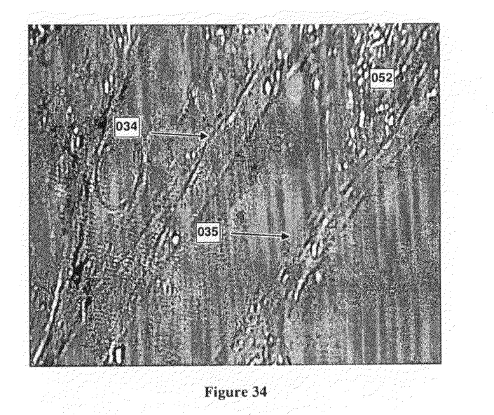

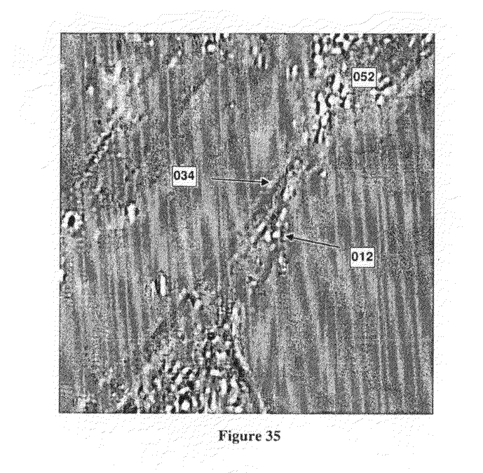

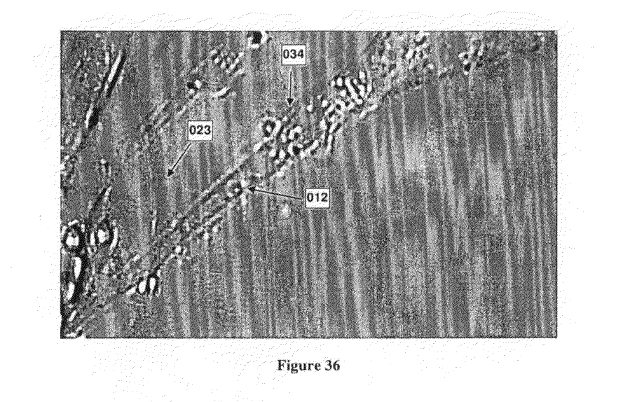

In FIG. 33, a frame from unstained live cell videography, tubules 034 and extensions 035 associated with TOS 012 activity in GBM 052 solid tumor are shown. TOS tubules 034 appear to mediate their formation by fusion at the outer edges of developing tubule 034a. TOS 012 can also migrate through tubules 034 from one aggregate of cells 064 to another (GBM tumor). In FIG. 34, another a frame from unstained live cell videography, TOS tubules 034 are shown emerging from cell body 052 as major extensions 035 serving as conduits for TOS migration. FIG. 35, another frame from unstained live cell videography, shows cell-to-cell associations mediated by TOS tubule interactions. Note an individual TOS 012 traveling through tubule 034 in GBM 052 tumor. In FIG. 36, another frame from unstained live cell videography, a TOS tubule 034 in association with the cell membrane 023 is shown in GBM 052 tumor cell.

In FIG. 21, TOS movements along ECM fibers produced by the tumor cells appeared to facilitate communication between different clusters of tumor cells associated with their organization to form larger masses via effects on cell mobility and migration. When TOS were observed to interact with patches of tumor cells already linked by cell-to-cell associations, the TOS association promoted the migration of these multi-cellular clusters of tumor cells to merge, thereby generating larger masses of tumor cell aggregates. The association of TOS with tumor cell aggregates appeared to continue until the,tumor cells were fully surrounded and in contact with neighboring cells, at which time the TOS frequently were observed to re-enter cells and become quiescent.

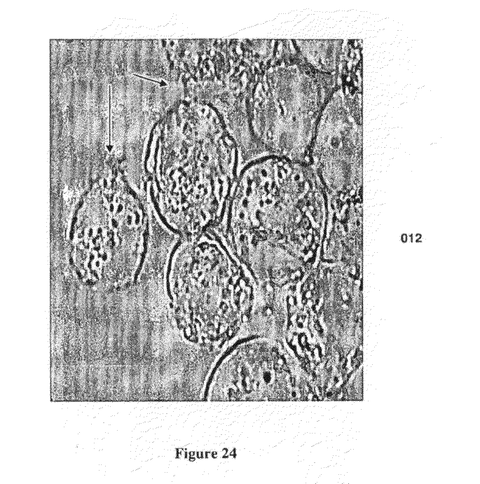

FIGS. 21a-i illustrate TOS bodies in solid tumor architecture. Solid tumors of Glioblastoma multiforme DBTRG.05MG were stained with periodic acid/Schiff reagent and photomicrographed (1000×) at 48 days in culture. FIG. 21a shows a GBM cell 062 in association with a peripheral structure formed by TOS 012. FIG. 21b is an example of highly organized tubule structure 034 formed by TOS 012 embedded in solid tumor. FIG. 21c shows TOS forming 018 in cell membrane of GBM solid tumor spheroid 038. FIG. 21d displays elements of solid tumor architecture showing highly tubular structures 034 formed by TOS in solid tumor GBM spheroids 038. FIGS. 21e-i illustrate TOS tubules structural details in association with TOS bodies at points of origin and internal structure. FIG. 21e shows solid tumor GBM edge 038 displaying unique morphology correlated with sites of TOS 018 formation. FIG. 21f is another view showing TOS structure 034 formation with solid tumor GBM 038. FIG. 21g is another view showing highly organized TOS geometric structural 034 formation within solid tumor GBM 038. FIG. 21h is another view showing TOS structure 034 formation with solid tumor GBM 038. FIG. 21i is a 4× low magnification of GBM solid tumor 038 in culture used as source of photomicroscopy images shown above at higher magnification. In addition, FIG. 24, a frame from live cell videography, shows TOS-mediated cell movement in NCI-H1299 lung carcinoma cells as indicated by the arrows.

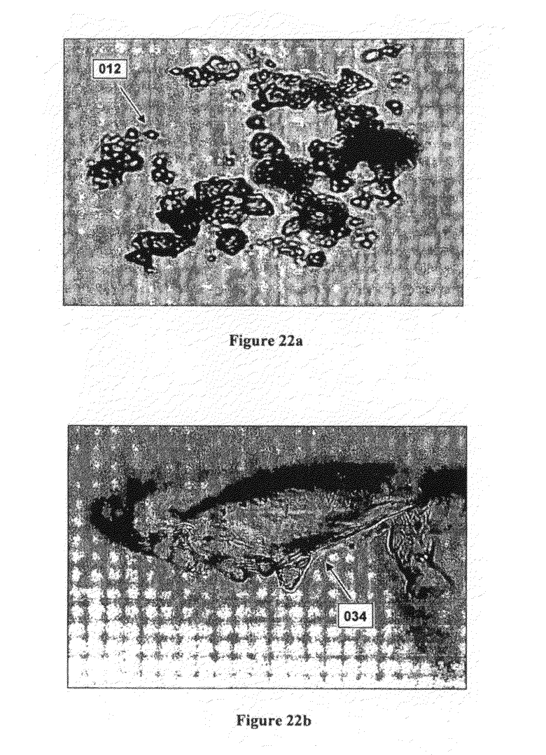

Studies of cell-free isolated TOS showed that these structures have the capability to reproduce and maintain long-term viability in culture as shown in FIG. 22. TOS were observed to generate complex tissue-like structures in vitro under these conditions. TOS structures were also observed to be produced in normal lung cell cultures (WI38) in vitro. FIGS. 22a-c are photomicrographs (1000×) showing TOS 012 isolated in culture. FIG. 22a is an H & E stained preparation showing cell free TOS 012 with arrow indicating single particle. FIG. 22b is an unstained photomicrograph shows structures 034 formed by TOS in cell free culture. FIG. 22c shows a scroll-like geometric structure 034 formed by TOS in cell free culture.

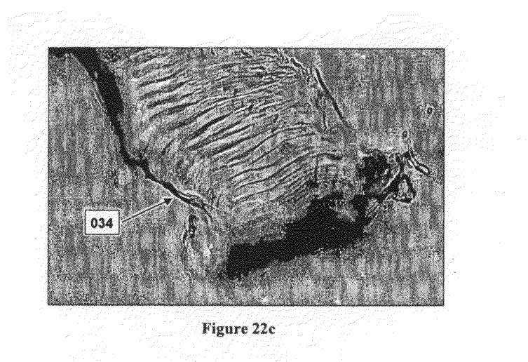

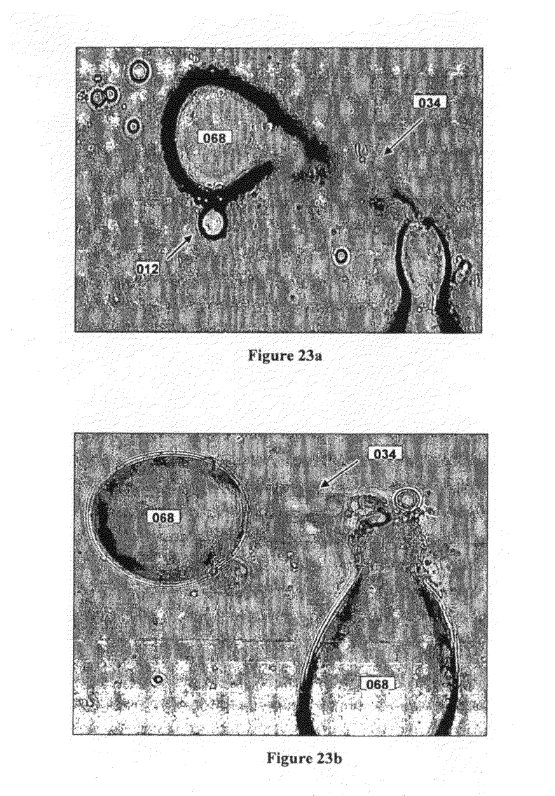

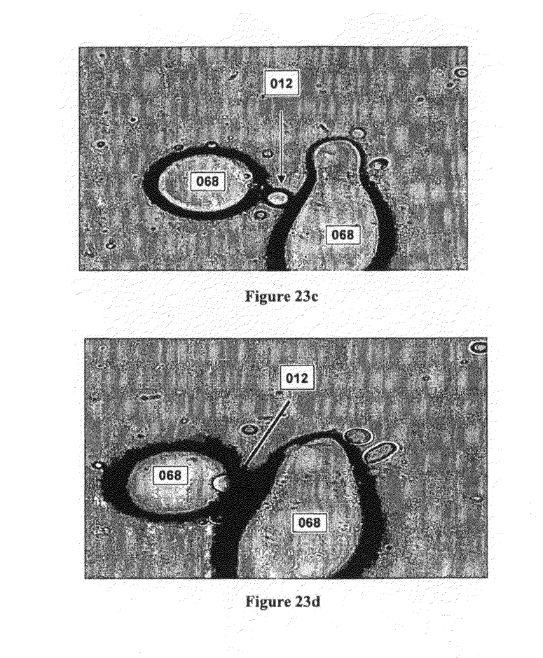

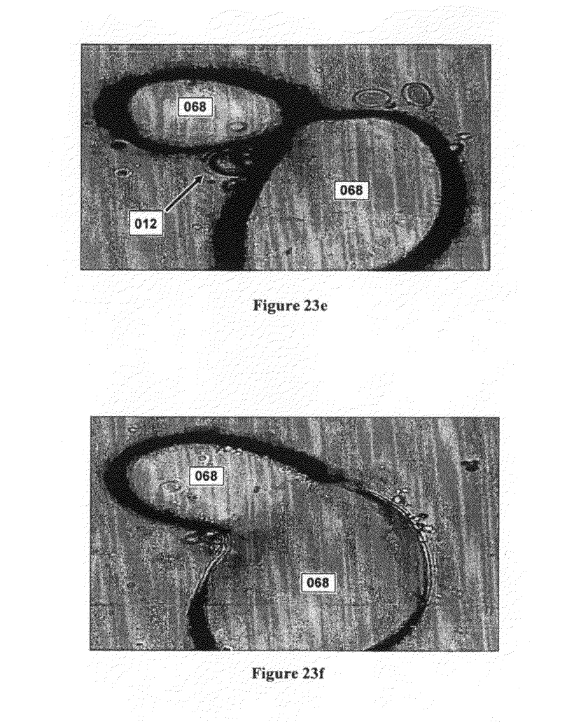

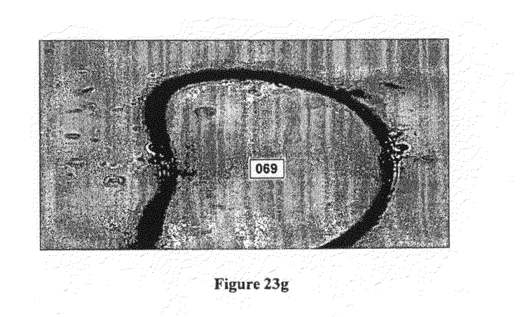

FIG. 23 is a series of live imaging photomicrographs (1000×) documenting the activities of cell-free TOS preparation in culture, merging and interacting to form larger structures. In FIGS. 23a and 23b, contact between large vesicles 068 formed by prior TOS particle fusion is established by tubular 034 projections. The sequence in FIGS. 23c-23g documents migration of TOS vesicles 068 which facilitates contact, then fusion of vesicles to form one large unit 069.

By utilizing TOS, an embodiment of a method of tissue engineering or tissue repair may comprise the steps of providing a source of tissue genetic material, culturing the tissue genetic material with an effective tissue organizing structure composition, isolating and purifying the tissue organization structure-tissue genetic material product, culturing the product with tissue target cells, promoting cellular differentiation of said target cells to said tissue specific phenotype activity, the differentiation being effective for said tissue repair.

An additional embodiment for producing cells with a tissue-specific phenotype useful for autogenous tissue repair comprises obtaining tissue from an intended patient, converting said patient tissue cells into repair/replacement cells by culturing said patient tissue cells with an effective tissue organizing structure composition, isolating and purifying the tissue organization structure-patient tissue product, culturing the product with tissue target cells, promoting cellular differentiation of said target cells to said patient tissue specific phenotype, the converted cells having effective patient tissue-specific phenotype activity.

An additional embodiment for a tissue repair precursor composition comprises a biocompatible matrix, a dense cell mass growing on the matrix and exposed to a culture medium, the dense cell mass being converted tissue-specific phenotype exhibiting cell appropriate morphology, the culture medium including a cellular inducing agent and at least one tissue organizing structure composition that promotes differentiation along the tissue-specific pathway.

### TOS Assessment in Genetic Engineering

TOS mediated genetic exchanges were observed following TOS fusion with tumor cells and were observed to produce rapid and dramatic morphogenetic alterations in tumor cell appearance, including distortions of size, nuclear to cytoplasmic ratio and surface asymmetries that in some cases were associated with the induction of tumor cell fission to generate tumor cells comprised entirely of TOS structures, that we have called ‘TOS bags” (see FIG. 3). Tumor cell fission to release large numbers of TOS appeared to be associated with tumor spread as TOS interacted with larger tumor cells, stimulated cell division and frequently were observed to organize to form cylindrical masses of TOS tubules (see FIG. 20).

TOS were observed to exist in one of two forms: either as ‘empty” membrane-bound vesicles or as vesicles containing genetic material. The genetic material was observed to be acquired by the empty vesicles following their entry into tumor cells, as this was often followed by their subsequent emergence with newly acquired contents comprised of genetic material.

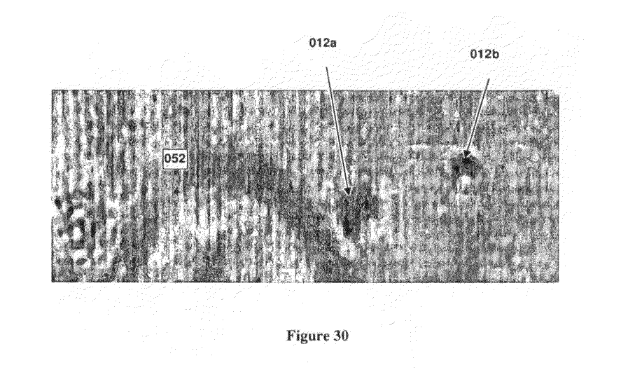

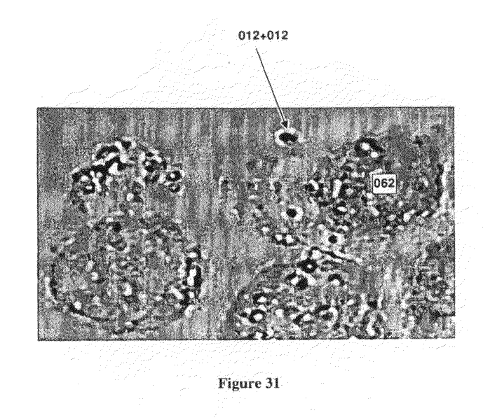

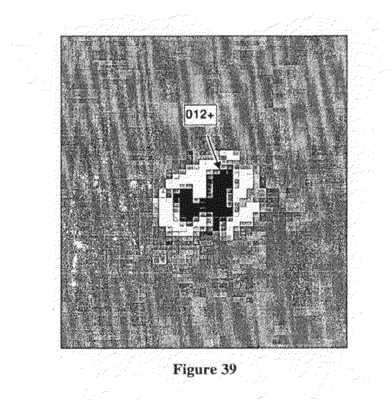

The TOS were also observed to fuse with one another and appeared to exchange genetic material before dissociating and moving apart. FIG. 30, a frame from live cell videography, shows TOS-TOS genetic exchange by fusion of two TOS 012a and 012b associated with chromosomal interactions (colon carcinoma cells SW620). In FIG. 31, a frame from live cell videography, TOS-cell associations with cell migration (NCI-H1299 lung carcinoma) and two TOS 012+012 fused with chromosome interactions are shown. FIG. 39 is a frame from unstained live cell videography showing fused TOS 012+ in genetic exchange. Note densely staining area in center suggesting genetic recombination 056 (MDA_MB_{—}231 breast carcinoma cells).

Overall, TOS were observed to be extraordinarily plastic with respect to their ability to fuse with one another to form doublets or even triplet complexes, and to enter tumor cells. These complexes form transiently and are frequently associated with genetic recombination. This plasticity extends to their genetic contents as well, in that TOS were observed to exchange genetic information with other TOS and also with tumor cells.

The observed activities of TOS demonstrate their potential utility in genetic engineering. TOS have been shown to be capable of inducing stable genetic phenotypic transformation of cultured cells. This capacity could be exploited to introduce therapeutic genes into target recipient cells. Moreover, the observed capacity of TOS to induce rapid morphogenetic conversion at the cellular level implicates these structures as potentially valuable tools to facilitate tissue transformations for therapeutic purposes

It is understood that the embodiments and descriptions herein are merely instruments of the application of the utility of TOS and those skilled in the art should realize that changes may be made without departure from the essential elements and contributions to the art as taught herein.

What is claimed is:

1. A composition comprising at least one tissue organizing structure and a diluent.

2. The composition of claim 1 wherein said diluent is culture medium.

3. The composition of claim 1 wherein said tissue organizing structure is of eukaryotic origin.

4. The composition of claim 1 wherein said tissue organizing structure is of mammal origin.

5. The composition of claim 1 wherein said tissue organizing structure is of human origin.

6. The composition of claim 1 wherein said tissue organizing structure is of plant origin.

7. The composition of claim 1 capable of being internalized with a target cell or tissue to achieve a desired effect.

8. A composition comprising at least one tissue organizing structure and a pharmaceutically acceptable carrier.

9. A pharmaceutical composition comprising purified tissue organizing structure having a therapeutic effective activity, combined in a mixture with a pharmaceutically acceptable carrier, to be administered to a patient in a therapeutically effective amount.

10. The composition of claim 9 wherein said pharmaceutically acceptable carrier is sterile phosphate buffered saline.

11. A method of isolating at least one tissue organizing structure comprising,
a. culturing cells overnight in serum-free culture medium to stimulate the production of tissue organizing structure in the cells,
b. removing the culture media,
c. washing the cells with a composition of phosphate buffered saline,
d. treating the cells with a composition of trypsin,
e. adding a composition of fetal bovine serum to the trypsanized cells,
f. incubating the cells,
g. releasing tissue organizing structure from the trypsanized cells,
h. binding the released tissue organizing structure to the culture dish,
i. removing the cells from the culture dish to isolate the bound tissue organizing structure.

12. The method of claim 11 wherein said cultured cells comprise normal diploid fibroblasts.

13. The method of claim 11 wherein said culture cells are derived from human cancer cell lines.

14. The method of claim 11 wherein said cells are treated with said trypsin composition for thirty minutes.

15. The method of claim 11 wherein said fetal bovine composition is a ten percent solution.

16. The method of claim 11 wherein said incubation is overnight at thirty-seven degrees centigrade.

17. A method of providing a purified composition of tissue organizing structure comprising,
a. culturing cells overnight in serum-free culture medium to stimulate the production of tissue organizing structure in the cells,
b. removing the culture media,
c. washing the cells with a composition of phosphate buffered saline,
d. treating the cells with a composition of trypsin,
e. adding a composition of fetal bovine serum to the trypsanized cells,
f. incubating the cells,
g. releasing tissue organizing structure from the trypsanized cells,
h. binding the released tissue organizing structure to the culture dish,
i. removing the cells from the culture dish to isolate the bound tissue organizing structure,
j. removing the tissue organizing structure from the culture dish with a cell scraper, and
k. resuspending the isolated tissue organizing structure in a diluent.

18. The method of claim 17 wherein said cultured cells comprise normal diploid fibroblasts.

19. The method of claim 17 wherein said culture cells are derived from human cancer cell lines.

20. The method of claim 17 wherein said cells are treated with said trypsin composition for thirty minutes.

21. The method of claim 17 wherein said fetal bovine composition is a ten percent solution.

22. The method of claim 17 wherein said incubation is overnight at thirty-seven degrees centigrade.

23. The method of claim 17 wherein said diluent comprises support for tissue organizing structure activity

24. The method of claim 17 wherein said diluent is culture medium.

25. A composition of tissue organizing structure capable of being internalized with a target cell or tissue to achieve a desired effect made by the process of culturing cells overnight in serum-free culture medium to stimulate the production of tissue organizing structure in the cells, removing the culture media, washing the cells with a composition of phosphate buffered saline, treating the cells with a composition of trypsin, adding a composition of ten percent fetal bovine serum to the trypsanized cells, incubating the cells, releasing tissue organizing structure from the trypsanized cells, binding the released tissue organizing structure to the culture dish, removing the cells from the culture dish to isolate the bound tissue organizing structure, removing the tissue organizing structure from the culture dish with a cell scraper, resuspending the isolated tissue organizing structure in a an acceptable diluent to substantially maintain tissue organizing structure activity in the composition, and introducing the composition to the target cell or tissue.

26. A method for inducing solid tumor formation comprising plating isolated cells on culture dishes coated with 1% agarose to block cell-to-substrate attachment in order to promote cell-to-cell interactions required for solid tumor formation.

27. The method of claim 26 wherein said culture cells comprise tumor cells derived from human cancer cell lines.

28. A method of identifying tissue organizing structure comprising staining tumor tissue with either hematoxylin/eosin, or periodic acid Schiff reagent/hematoxylin, visualizing stained tissue organizing structure by microscopy, and identifying said tissue organizing structure.

29. The method of claim 28 wherein said tissue organizing structure comprises an approximate diameter of 2 microns.

30. The method of claim 28 wherein the exterior surface of said tissue organizing structure comprises lipid.

31. The method of claim 28 wherein the interior of said tissue organizing structure comprises genetic material.

32. The method of claim 28 wherein the interior of said tissue organizing structure comprises DNA.

33. A method of tissue organizing structure formation comprising inducing a cell to abrogate stromal attachment in the tissue of origin, initiating cell membrane changes, producing a tissue organizing structure bud in said membrane, releasing said tissue organizing structure from said bud.

34. A method of tissue organizing structure formation comprising fusion of said tissue organizing structure with a cell, initiating intracellular production of a plurality of tissue organizing structures, releasing said tissue organizing structures from said cell by fission.

35. A method of inducing malignant transformation of normal cells comprising the steps of,
a. culturing cells overnight in serum-free culture medium to stimulate the production of tissue organizing structure in the cells,
b. removing the culture media,
c. washing the cells with a composition of phosphate buffered saline,
d. treating the cells with a composition of trypsin,
e. adding a composition of fetal bovine serum to the trypsanized cells,
f. incubating the cells,
g. releasing tissue organizing structure from the trypsanized cells,
h. binding the released tissue organizing structure to the culture dish,
i. removing the cells from the culture dish to isolate the bound tissue organizing structure.
j. suspending tissue organizing structure in phosphate buffered saline solution,
k. adding the tissue organizing structure suspension to a culture dish containing a monolayer of normal cells growing in culture medium,
l. incubating the tissue organizing structure treated cells at thirty seven degrees centigrade in five percent carbon dioxide, and
m. producing tissue organizing structure transformed malignant cells comprising the phenotype of the source malignant cells.

36. The method of claim 35 wherein the culture cells are carcinoma cells.

37. The method of claim 35 wherein the culture cells comprise approximately 2×10̂5 cells.

38. The method of claim 35 wherein the normal cells are lung fibroblasts.

39. The method of claim 35 wherein the monolayer of normal cells comprises approximately 2×10̂5 cells.

40. A method of phenotypic reversion in a cell comprising,
a. Providing a tissue organizing composition derived from normal functioning cells,
b. Adding said composition to a culture of malignant cells,
c. incubating said culture,
d. observing phenotypic reversion of the malignant cell to the phenotype of the normal cell.

41. The method of claim 40 wherein said normal cells comprise approximately 1×10̂4 cells and said malignant cells comprise 1×10̂6 cells.

42. A method of inducing death in a tumor cell comprising providing a tissue organizing composition derived from a concentration of approximately 1×10̂4 normal functioning cells, contacting said composition with a culture of approximately 1×10̂6 malignant cells, incubating said culture, and inducing cell death.

43. The method of claim 42 wherein said tissue organizing structures are derived from a plant source.

44. The method of claim 42 wherein said malignant cells are human.

45. The method of claim 42 wherein the malignant cells are selected from the group consisting of leukemia cells, non-small cell lung cancer cells, colon cancer cells, central nervous system cancer cells, melanoma cells, ovarian cancer cells, renal cancer cells, ovarian cancer cells, prostate cancer cells, and breast cancer cells.

46. A method of treating cancer cells wherein the cancer cells are selected from the group consisting of leukemia, cells, non-small cell lung cancer cells, colon cancer cells, central nervous system cancer cells, melanoma cells, ovarian cancer cells, renal cancer cells, ovarian cancer cells, prostate cancer cells, and breast cancer cells, said method comprising administering to the cancer cells a cancer cell-treating effective amount of a composition of tissue organizing structure.

47. A method of treating a disorder in a mammal, said disorder being characterized by hyperplastic cell growth, comprising administering to said mammal a therapeutically effective amount of a tissue organizing structure composition.

48. A method of tissue engineering or tissue repair comprising the steps of providing a source of tissue genetic material, culturing the tissue genetic material with an effective tissue organizing structure composition, isolating and purifying the tissue organization structure-tissue genetic material product, culturing the product with tissue target cells, promoting cellular differentiation of said target cells to said tissue specific phenotype activity, the differentiation being effective for said tissue repair.

49. A method for producing cells with a tissue-specific phenotype useful for autogenous tissue repair comprising, obtaining tissue from an intended patient, converting said patient tissue cells into repair/replacement cells by culturing said patient tissue cells with an effective tissue organizing structure composition, isolating and purifying the tissue organization structure-patient tissue product, culturing the product with tissue target cells, promoting cellular differentiation of said target cells to said patient tissue specific phenotype, the converted cells having effective patient tissue-specific phenotype activity.

50. A tissue repair precursor composition comprising a biocompatible matrix, and a dense cell mass growing on the matrix and exposed to a culture medium, the dense cell mass being converted tissue-specific phenotype exhibiting cell appropriate morphology, the culture medium including a cellular inducing agent and at least one tissue organizing structure composition that promotes differentiation along the tissue-specific pathway.

51. A method of transferring genetic material between cells comprising,
a. selecting a plurality of cells from a patient or other source having genetic material expressing specific phenotype characteristics,
b. transferring said specific genetic material to a plurality of tissue organizing structures by incubating said selected cells with a tissue organizing structure composition,
c. isolating and purifying said tissue organizing structure-genetic material from said selected cell culture,
d. suspending said tissue organizing structure-genetic material in phosphate buffered saline solution,
e. adding said tissue organizing structure-genetic material solution to a culture of target cells,
f. incubating the culture,
g. producing transformed target cells comprising the phenotype of the source cells.
